(19) United States
(12) Patent Application Publication
(10) Pub. No.: US 2012/0174246 A1
(43) Pub. Date: Jul. 5, 2012

(54) METHODS OF IDENTIFYING APHID RESISTANT SOYBEANS

(76) Inventors: Julian M. Chaky, Urbandale, IA (US); Molly Ryan-Mahmutagic, Waukee, IA (US); Edwin Josue Mendez, West Des Moines, IA (US); Sally Anne Santiago-Parton, Ankeny, IA (US); Joshua M. Shendelman, Ankeny, IA (US); John B. Woodward, Ankeny, IA (US); Yanwen Xiong, Johnston, IA (US)

(21) Appl. No.: 13/339,548

(22) Filed: Dec. 29, 2011

Related U.S. Application Data
(60) Provisional application No. 61/428,306, filed on Dec. 30, 2010.

(51) Int. Cl. A01H 1/02; C12Q 1/68; C07H 21/04; A01H 1/00

(52) U.S. Cl. 800/260; 800/278; 435/6.12; 536/23.1

(57) ABSTRACT

This invention relates to methods of identifying and/or selecting soybean plants or germplasm that display improved antibiosis and/or antixenosis resistance to one or more biotypes of soybean aphid. In certain examples, the method comprises detecting at least one Rag haplotype that is associated with improved soybean aphid resistance. In other examples, the method further comprises detecting a marker profile comprising two or more Rag haplotypes.

### CROSS-REFERENCE TO RELATED APPLICATIONS

This application claims priority to and benefit of U.S. Provisional Patent Application Ser. No. 61/428,306, filed on Dec. 30, 2010, which is hereby incorporated by reference in its entirety.

### FIELD OF THE INVENTION

This invention relates to methods of identifying and/or selecting soybean plants or germplasm that display improved antibiosis and/or antixenosis resistance to one or more biotypes of soybean aphid.

### BACKGROUND

Soybeans (Glycine max L. Merr.) are a major cash crop and investment commodity in North America and elsewhere. Soybean oil is one of the most widely used edible oils, and soybeans are used worldwide both in animal feed and in human food production. Additionally, soybean utilization is expanding to industrial, manufacturing, and pharmaceutical applications. Soybeans are also vulnerable to more than one hundred different pathogens, with some pathogens having disastrous economic consequences. One important soybean pathogen is the soybean aphid, which can severely impact yield. Despite a large amount of effort expended in the art, commercial soybean crops are still largely susceptible to aphid infestation.

A native of Asia, the soybean aphid (Aphis glycines Matsumura) was first found in the Midwest in 2000 (Hartman, G. L., et al., “Occurrence and distribution of Aphis glycines on soybeans in Illinois in 2000 and its potential control,” (1 Feb. 2001), available at http://plantmanagementnetwork.org/phpldefault.asp). It rapidly spread throughout the region and into other parts of North America (Patterson, J. and Ragsdale, D., “Assessing and managing risk from soybean aphids in the North Central States,” (11 Apr. 2002) available at http://planthealthinfo/aphids_researchupdate.htm). High aphid populations can reduce crop production directly when their feeding causes severe damage such as stunting, leaf distortion, and reduced pod set (Sun, Z., et al., “Study on the uses of aphid-resistant character in wild soybean. I. Aphid-resistance performance of F2 generation from crosses between cultivated and wild soybeans,” (1990) Soybean Genet. News. 17:43-48). Yield losses attributed to the aphid in some fields in Minnesota during 2001, where several thousand aphids occurred on individual soybean plants, were >50% (Ostlie, K., “Managing soybean aphid,” (2 Oct. 2002), available at http://soybeans.umn.edu/crop/insects/aphid/aphid˜publicationmanagingsba.htm), with an average loss of 101 to 202 kg/ha in those fields (Patterson, J. and Ragsdale, D., “Assessing and managing risk from soybean aphids in the North Central States,” (11 Apr. 2002). In earlier reports from China, soybean yields were reduced up to 52% when there was an average of about 220 aphids per plant (Wang, X. B., et al., “A study on the damage and economic threshold of the soybean aphid at the seedling stage,” (1994) Plant Prot. (China) 20:12-13), and plant height was decreased by about 210 mm after severe aphid infestation (Wang, X. B., et al., “Study on the effects of the population dynamics of soybean aphid (Aphis glycines) on both growth and yield of soybean,” (1996) Soybean Sci. 15:243-247). An additional threat posed by the aphid is its ability to transmit certain plant viruses to soybean, such as Alfalfa mosaic virus, Soybean dwarf virus, and Soybean mosaic virus (Sama, S., et al., “Varietal screening for resistance to the aphid, Aphis glycines, in soybean,” (1974) Research Reports 1968-1974, pp. 171-172; Iwaki, M., et al., “A persistent aphid borne virus of soybean, Indonesian Soybean dwarf virus transmitted by Aphis glycines,” (1980) Plant Dis. 64:1027-1030; Hartman, G. L., et al., “Occurrence and distribution of Aphis glycines on soybeans in Illinois in 2000 and its potential control,” (1 Feb. 2001), available at http://plantmanagementnetwork.org/phpldefault.asp; Hill, J. H., et al., “First report of transmission of Soybean mosaic virus and Alfalfa mosaic virus by Aphis glycines (Homoptera, Aphididae),” (1996) Appl. Entomol. 2001. 31:178-180; Clark, A. J. and Perry, K. L., “Transmissibility of field isolates of soybean viruses by Aphis glycines,” (2002) Plant Dis. 86:1219-1222).

Currently, millions of dollars are spent annually on spraying insecticides to control soybean aphid infestation. An integral component of an integrated pest management (IPM) program to control aphids is plant resistance (Auclair, J. L., “Host plant resistance,” pp. 225-265 In P. Harrewijn (ed.) Aphids: Their biology, natural enemies, and control, Vol. C., Elsevier, New York (1989); Harrewijn, P. and Minks, A. K., “Integrated aphid management: General aspects,” pp. 267-272, In A. K. Minks and P. Harrewijn (ed.) Aphids: Their biology, natural enemies, and control, Vol. C., Elsevier, New York (1989)). Insect resistance can significantly reduce input costs for producers (Luginbill, J. P., “Developing resistant plants—The ideal method of controlling insects,” (1969) USDA, ARS. Prod. Res. Rep. 111, USGPO, Washington, D.C.).

There are currently three well-documented biotypes (i.e., a subspecies of soybean aphid that shares certain genetic traits or a specified genotype) of soybean aphid that have been collected in Urbana, Ill. (biotype 1), Wooster, Ohio (biotype 2), and Indiana (biotype 3). Additionally, there are three kinds of plant resistance that have been identified: antibiosis, antixenosis, and tolerance. Antibiosis (non-choice) is the plant's ability to reduce the survival, reproduction, and fecundity of the insect. Antixenosis (choice) is the plant's ability to deter the insect from feeding or identifying the plant as a food source. Tolerance is the plant's ability to withstand heavy infestation without significant yield loss.

To date, three different soybean aphid resistance genes have been identified and mapped to the soybean genome. Rag1 was the first soybean resistance gene identified (Mian, et al., Genetic linkage mapping of the soybean aphid resistance gene in PI 243540, Theor. Appl. Genet. 117:955-962 (2008)). Rag1 has been mapped to linkage group M in the vicinity of SSR markers Satt540 and Satt463 (Kim, et al., Fine mapping of the soybean aphid resistance gene Rag1 in soybean, Theor. Appl. Genet., 120:1063-1071 (2010)). Rag2 has been mapped to linkage group F in the vicinity of SSR markers Satt334 and Sct_{—}033 (Mian, et al., Genetic linkage mapping of the soybean aphid resistance gene in PI 243540, Theor. Appl. Genet. 117:955-962 (2008)). Rag3 is located on linkage group J in the vicinity of markers Sat_{—}339 and Sat_{—}370. It has also been previously determined that some aphid biotypes are resistant to certain of the Rag genes but are susceptible to others (Mian, et al., Genetic linkage mapping of the soybean aphid resistance gene in PI 243540, Theor. Appl. Genet. 117:955-962 (2008)).

Molecular markers have been used to selectively improve soybean crops through the use of marker assisted selection. Any detectable polymorphic trait can be used as a marker so long as it is inherited differentially and exhibits linkage disequilibrium with a phenotypic trait of interest A number of soybean markers have been mapped and linkage groups created, as described in Cregan, P. B., et al., “An Integrated Genetic Linkage Map of the Soybean Genome” (1999) Crop Science 39:1464-90, and more recently in Choi, et al., “A Soybean Transcript Map: Gene Distribution, Haplotype and Single-Nucleotide Polymorphism Analysis” (2007) Genetics 176:685-96. Many soybean markers are publicly available at the USDA affiliated soybase website (www.soybase.org).

Most plant traits of agronomic importance are polygenic, otherwise known as quantitative, traits. A quantitative trait is controlled by several genes located at various locations, or loci, in the plant's genome. The multiple genes have a cumulative effect which contributes to the continuous range of phenotypes observed in many plant traits. These genes are referred to as quantitative trait loci (QTL). Recombination frequency measures the extent to which a molecular marker is linked with a QTL. Lower recombination frequencies, typically measured in centiMorgans (cM), indicate greater linkage between the QTL and the molecular marker. The extent to which two features are linked is often referred to as the genetic distance. The genetic distance is also typically related to the physical distance between the marker and the QTL; however, certain biological phenomenon (including recombinational “hot spots”) can affect the relationship between physical distance and genetic distance. Generally, the usefulness of a molecular marker is determined by the genetic and physical distance between the marker and the selectable trait of interest.

In some cases, multiple closely linked markers, such as Single Nucleotide Polymorphism (SNP) markers, can be found to exist in a certain region of a plant genome encompassing one or more QTL. In such cases, by determining the allele present at each of those marker loci, a haplotype for that region of the plant genome can be determined. Further, by determining alleles or haplotypes present at multiple regions of the plant genome related to the same phenotypic trait, a marker profile for that trait can be determined. Such haplotype and marker profile information can be useful in identifying and selecting plants with certain desired traits.

There remains a need for soybean plants with improved resistance to soybean aphid and methods for identifying and selecting such plants.

### SUMMARY

This invention relates to methods of identifying and/or selecting soybean plants or germplasm that display improved antibiosis and/or antixenosis resistance to one or more biotypes of soybean aphid. In certain examples, the method comprises detecting at least one Rag haplotype that is associated with improved soybean aphid resistance. In other examples, the method further comprises detecting a marker profile comprising two or more Rag haplotypes. In further examples, the method further comprises crossing a selected soybean plant with a second soybean plant. This invention further relates to markers, primers, probes, kits, systems, etc., useful for carrying out the methods described herein.

### BRIEF DESCRIPTION OF THE FIGURES

FIGS. 1A-1C illustrate a partial genetic map of soybean illustrating the relative map position of the Rag intervals and numerous linked marker loci. FIG. 1A illustrates a genetic map of linkage group M and the relative map position of the Rag1 interval. FIG. 1B illustrates a genetic map of linkage group F and the relative map position of the Rag2 interval. FIG. 1C illustrates a genetic map of linkage group J and the relative map position of the Rag3 interval.

### SUMMARY OF THE SEQUENCES

SEQ ID NOs: 1-4 comprise nucleotide sequences of regions of the soybean genome, each capable of being used as a probe or primer, either alone or in combination, for the detection of marker locus S14181-1-Q1 on LG-M. In certain examples, SEQ ID NOs: 1 and 2 are used as primers while SEQ ID NOs: 3 and 4 are used as probes.

SEQ ID NO: 5 is the genomic DNA region encompassing marker locus S14181-1-Q1 on LG-M. In certain examples this sequence is used to design primers and probes directed toward this marker. In certain other examples this sequence, or a portion of it, is used as a probe to detect this marker.

SEQ ID NOs: 6-9 comprise nucleotide sequences of regions of the soybean genome, each capable of being used as a probe or primer, either alone or in combination, for the detection of marker locus S13871-1-Q1 on LG-M. In certain examples, SEQ ID NOs: 6 and 7 are used as primers while SEQ ID NOs: 8 and 9 are used as probes.

SEQ ID NO: 10 is the genomic DNA region encompassing marker locus S13871-1-Q1 on LG-M. In certain examples this sequence is used to design primers and probes directed toward this marker. In certain other examples this sequence, or a portion of it, is used as a probe to detect this marker.

SEQ ID NOs: 11-14 comprise nucleotide sequences of regions of the soybean genome, each capable of being used as a probe or primer, either alone or in combination, for the detection of marker locus S14161-1-Q10 on LG-M. In certain examples, SEQ ID NOs: 11 and 12 are used as primers while SEQ ID NOs: 13 and 14 are used as probes.

SEQ ID NO: 15 is the genomic DNA region encompassing marker locus S14161-1-Q10 on LG-M. In certain examples this sequence is used to design primers and probes directed toward this marker. In certain other examples this sequence, or a portion of it, is used as a probe to detect this marker.

SEQ ID NOs: 16-19 comprise nucleotide sequences of regions of the soybean genome, each capable of being used as a probe or primer, either alone or in combination, for the detection of marker locus S09515-1-Q1 on LG-M. In certain examples, SEQ ID NOs: 16 and 17 are used as primers while SEQ ID NOs: 18 and 19 are used as probes.

SEQ ID NO: 20 is the genomic DNA region encompassing marker locus S09515-1-Q1 on LG-M. In certain examples this sequence is used to design primers and probes directed toward this marker. In certain other examples this sequence, or a portion of it, is used as a probe to detect this marker.

SEQ ID NOs: 21-24 comprise nucleotide sequences of regions of the soybean genome, each capable of being used as a probe or primer, either alone or in combination, for the detection of marker locus S14151-1-Q1 on LG-M. In certain examples, SEQ ID NOs: 21 and 22 are used as primers while SEQ ID NOs: 23 and 24 are used as probes.

SEQ ID NO: 25 is the genomic DNA region encompassing marker locus S14151-1-Q1 on LG-M. In certain examples this sequence is used to design primers and probes directed toward this marker. In certain other examples this sequence, or a portion of it, is used as a probe to detect this marker.

SEQ ID NOs: 26-29 comprise nucleotide sequences of regions of the soybean genome, each capable of being used as a probe or primer, either alone or in combination, for the detection of marker locus S14151-2-Q4 on LG-M. In certain examples, SEQ ID NOs: 26 and 27 are used as primers while SEQ ID NOs: 28 and 29 are used as probes.

SEQ ID NO: 30 is the genomic DNA region encompassing marker locus S14151-2-Q4 on LG-M. In certain examples this sequence is used to design primers and probes directed toward this marker. In certain other examples this sequence, or a portion of it, is used as a probe to detect this marker.

SEQ ID NOs: 31-34 comprise nucleotide sequences of regions of the soybean genome, each capable of being used as a probe or primer, either alone or in combination, for the detection of marker locus S07164-1-Q12 on LG-M. In certain examples, SEQ ID NOs: 31 and 32 are used as primers while SEQ ID NOs: 33 and 34 are used as probes.

SEQ ID NO: 35 is the genomic DNA region encompassing marker locus S07164-1-Q12 on LG-M. In certain examples this sequence is used to design primers and probes directed toward this marker. In certain other examples this sequence, or a portion of it, is used as a probe to detect this marker.

SEQ ID NOs: 36-39 comprise nucleotide sequences of regions of the soybean genome, each capable of being used as a probe or primer, either alone or in combination, for the detection of marker locus S14182-1-Q1 on LG-M. In certain examples, SEQ ID NOs: 36 and 37 are used as primers while SEQ ID NOs: 38 and 39 are used as probes.

SEQ ID NO: 40 is the genomic DNA region encompassing marker locus S14182-1-Q1 on LG-M. In certain examples this sequence is used to design primers and probes directed toward this marker. In certain other examples this sequence, or a portion of it, is used as a probe to detect this marker.

SEQ ID NOs: 41-44 comprise nucleotide sequences of regions of the soybean genome, each capable of being used as a probe or primer, either alone or in combination, for the detection of marker locus S00812-1-A on LG-M. In certain examples, SEQ ID NOs: 41 and 42 are used as primers while SEQ ID NOs: 43 and 44 are used as probes.

SEQ ID NO: 45 is the genomic DNA region encompassing marker locus S00812-1-A on LG-M. In certain examples this sequence is used to design primers and probes directed toward this marker. In certain other examples this sequence, or a portion of it, is used as a probe to detect this marker.

SEQ ID NOs: 46-49 comprise nucleotide sequences of regions of the soybean genome, each capable of being used as a probe or primer, either alone or in combination, for the detection of marker locus S02780-1-A on LG-M. In certain examples, SEQ ID NOs: 46 and 47 are used as primers while SEQ ID NOs: 48 and 49 are used as probes.

SEQ ID NO: 50 is the genomic DNA region encompassing marker locus S02780-1-A on LG-M. In certain examples this sequence is used to design primers and probes directed toward this marker. In certain other examples this sequence, or a portion of it, is used as a probe to detect this marker.

SEQ ID NOs: 51-54 comprise nucleotide sequences of regions of the soybean genome, each capable of being used as a probe or primer, either alone or in combination, for the detection of marker locus S14761-00′-Q001 on LG-F. In certain examples, SEQ ID NOs: 51 and 52 are used as primers while SEQ ID NOs: 53 and 54 are used as probes.

SEQ ID NO: 55 is the genomic DNA region encompassing marker locus S14761-001-Q001 on LG-F. In certain examples this sequence is used to design primers and probes directed toward this marker. In certain other examples this sequence, or a portion of it, is used as a probe to detect this marker.

SEQ ID NOs: 56-59 comprise nucleotide sequences of regions of the soybean genome, each capable of being used as a probe or primer, either alone or in combination, for the detection of marker locus S14771-001-Q001 on LG-F. In certain examples, SEQ ID NOs: 56 and 57 are used as primers while SEQ ID NOs: 58 and 59 are used as probes.

SEQ ID NO: 60 is the genomic DNA region encompassing marker locus S14771-001-Q001 on LG-F. In certain examples this sequence is used to design primers and probes directed toward this marker. In certain other examples this sequence, or a portion of it, is used as a probe to detect this marker.

SEQ ID NOs: 61-64 comprise nucleotide sequences of regions of the soybean genome, each capable of being used as a probe or primer, either alone or in combination, for the detection of marker locus S07165-1-Q3 on LG-F. In certain examples, SEQ ID NOs: 61 and 62 are used as primers while SEQ ID NOs: 63 and 64 are used as probes.

SEQ ID NO: 65 is the genomic DNA region encompassing marker locus S07165-1-Q3 on LG-F. In certain examples this sequence is used to design primers and probes directed toward this marker. In certain other examples this sequence, or a portion of it, is used as a probe to detect this marker.

SEQ ID NOs: 66-69 comprise nucleotide sequences of regions of the soybean genome, each capable of being used as a probe or primer, either alone or in combination, for the detection of marker locus S14778-001-Q001 on LG-F. In certain examples, SEQ ID NOs: 66 and 67 are used as primers while SEQ ID NOs: 68 and 69 are used as probes.

SEQ ID NO: 70 is the genomic DNA region encompassing marker locus S14778-001-Q001 on LG-F. In certain examples this sequence is used to design primers and probes directed toward this marker. In certain other examples this sequence, or a portion of it, is used as a probe to detect this marker.

SEQ ID NOs: 71-74 comprise nucleotide sequences of regions of the soybean genome, each capable of being used as a probe or primer, either alone or in combination, for the detection of marker locus S01164-1-Q1 on LG-F. In certain examples, SEQ ID NOs: 71 and 72 are used as primers while SEQ ID NOs: 73 and 74 are used as probes.

SEQ ID NO: 75 is the genomic DNA region encompassing marker locus S01164-1-Q1 on LG-F. In certain examples this sequence is used to design primers and probes directed toward this marker. In certain other examples this sequence, or a portion of it, is used as a probe to detect this marker.

SEQ ID NOs: 76-83 comprise nucleotide sequences of regions of the soybean genome, each capable of being used as a probe or primer, either alone or in combination, for the detection of marker locus S13662-1-Q3/Q6 on LG-J. In certain examples, SEQ ID NOs: 76 and 77 are used as primers while SEQ ID NOs: 78 and 79 are used as probes to amplify and detect S13662-1-Q3. In other examples, SEQ ID NOs: 80 and 81 are used as primers while SEQ ID NOs: 82 and 83 are used as probes to amplify and detect S13662-1-Q6.

SEQ ID NO: 84 is the genomic DNA region encompassing marker locus S13662-1-Q3/Q6 on LG-J. In certain examples this sequence is used to design primers and probes directed toward this marker. In certain other examples this sequence, or a portion of it, is used as a probe to detect this marker.

SEQ ID NOs: 85-88 comprise nucleotide sequences of regions of the soybean genome, each capable of being used as a probe or primer, either alone or in combination, for the detection of marker locus S13663-1-Q1 on LG-J. In certain examples, SEQ ID NOs: 85 and 86 are used as primers while SEQ ID NOs: 87 and 88 are used as probes.

SEQ ID NO: 89 is the genomic DNA region encompassing marker locus S13663-1-Q1 on LG-J. In certain examples this sequence is used to design primers and probes directed toward this marker. In certain other examples this sequence, or a portion of it, is used as a probe to detect this marker.

SEQ ID NOs: 90-93 comprise nucleotide sequences of regions of the soybean genome, each capable of being used as a probe or primer, either alone or in combination, for the detection of marker locus S11411-1-Q1 on LG-J. In certain examples, SEQ ID NOs: 90 and 91 are used as primers while SEQ ID NOs: 92 and 93 are used as probes.

SEQ ID NO: 94 is the genomic DNA region encompassing marker locus S11411-1-Q1 on LG-J. In certain examples this sequence is used to design primers and probes directed toward this marker. In certain other examples this sequence, or a portion of it, is used as a probe to detect this marker.

SEQ ID NOs: 95-102 comprise nucleotide sequences of regions of the soybean genome, each capable of being used as a probe or primer, either alone or in combination, for the detection of marker locus S13664-1-Q1/Q002 on LG-J. In certain examples, SEQ ID NOs: 95 and 96 are used as primers while SEQ ID NOs: 97 and 98 are used as probes to amplify and detect S13664-1-Q1. In other examples, SEQ ID NOs: 99 and 100 are used as primers while SEQ ID NOs: 101 and 102 are used as probes to amplify and detect S13664-1-Q002.

SEQ ID NO: 103 is the genomic DNA region encompassing marker locus S13664-1-Q002 on LG-J. In certain examples this sequence is used to design primers and probes directed toward this marker. In certain other examples this sequence, or a portion of it, is used as a probe to detect this marker.

SEQ ID NOs: 104-113 comprise nucleotide sequences of regions of the soybean genome, each capable of being used as a probe or primer, either alone or in combination, for the detection of marker locus S13672-1-Q1/Q2/Q3 on LG-J. In certain examples, SEQ ID NOs: 104 and 105 are used as primers while SEQ ID NOs: 106 and 107 are used as probes to amplify and detect S13672-1-Q1. In other examples, SEQ ID NOs: 108 and 109 are used as primers while SEQ ID NOs: 106 and 107 are used as probes to amplify and detect S13672-1-Q2. In still further examples, SEQ ID NOs: 110 and 111 are used as primers while SEQ ID NOs: 112 and 113 are used as probes to amplify and detect S13672-1-Q3.

SEQ ID NO: 114 is the genomic DNA region encompassing marker locus S13672-1-Q1/Q2/Q3 on LG-J. In certain examples this sequence is used to design primers and probes directed toward this marker. In certain other examples this sequence, or a portion of it, is used as a probe to detect this marker.

SEQ ID NOs: 115-120 comprise nucleotide sequences of regions of the soybean genome, each capable of being used as a probe or primer, either alone or in combination, for the detection of marker locus S13674-1-Q1/Q007 on LG-J. In certain examples, SEQ ID NOs: 115 and 116 are used as primers while SEQ ID NOs: 117 and 118 are used as probes to amplify and detect S13674-1-Q1. In other examples, SEQ ID NOs: 119 and 120 are used as primers while SEQ ID NOs: 117 and 118 are used as probes to amplify and detect S13674-1-Q007.

SEQ ID NO: 121 is the genomic DNA region encompassing marker locus S13674-1-Q1/Q007 on LG-J. In certain examples this sequence is used to design primers and probes directed toward this marker. In certain other examples this sequence, or a portion of it, is used as a probe to detect this marker.

SEQ ID NOs: 122-125 comprise nucleotide sequences of regions of the soybean genome, each capable of being used as a probe or primer, either alone or in combination, for the detection of marker locus S13675-2-Q1 on LG-J. In certain examples, SEQ ID NOs: 122 and 123 are used as primers while SEQ ID NOs: 124 and 125 are used as probes.

SEQ ID NO: 126 is the genomic DNA region encompassing marker locus S13675-2-Q1 on LG-J. In certain examples this sequence is used to design primers and probes directed toward this marker. In certain other examples this sequence, or a portion of it, is used as a probe to detect this marker.

SEQ ID NOs: 127-130 comprise nucleotide sequences of regions of the soybean genome, each capable of being used as a probe or primer, either alone or in combination, for the detection of marker locus S01190-1-A on LG-F. In certain examples, SEQ ID NOs: 127 and 128 are used as primers while SEQ ID NOs: 129 and 130 are used as probes.

SEQ ID NO: 131 is the genomic DNA region encompassing marker locus S01190-1-A on LG-F. In certain examples this sequence is used to design primers and probes directed toward this marker. In certain other examples this sequence, or a portion of it, is used as a probe to detect this marker.

### DETAILED DESCRIPTION

A novel method is provided for identifying a soybean plant or germplasm that displays improved resistance to one or more aphid biotypes, the method comprising detecting in the soybean plant or germplasm, or a part thereof, at least one Rag marker or haplotype that is associated with improved soybean aphid resistance. In certain examples, the improved resistance comprises one or more of improved antibiosis resistance and improved antixenosis resistance. In other examples, the improved resistance comprises both improved antibiosis resistance and improved antixenosis resistance. In other examples, the improved soybean aphid resistance comprises improved resistance to at least two soybean aphid biotypes. In still other examples, the improved soybean aphid resistance comprises improved resistance to three or all four of soybean aphid biotypes 1, 2, 3, and X.

In certain examples, the at least one Rag haplotype is a favorable haplotype that positively correlates with improved soybean aphid resistance. In other examples, the at least one Rag haplotype is a disfavorable haplotype that negatively correlates with improved soybean aphid resistance.

In some examples, the Rag1 haplotype comprises one or more markers that fall within the interval flanked by and including Satt435 and Sat_{—}244, the Rag2 haplotype comprises one or more markers that fall within the interval flanked by and including Satt334 and Satt510, and/or the Rag3 haplotype comprises one or more markers that fall within the interval flanked by and including Sat_{—}339 and Sat_{—}370. In other examples, the Rag1 haplotype comprises one or more markers that fall within the interval flanked by and including physical position 5464314-8194502 on LG-M on the Glyma1 soybean genome assembly, the Rag2 haplotype comprises one or more markers that fall within the interval flanked by and including physical position 28416122-30590233 on LG-F on the Glyma1 soybean genome assembly, and/or the Rag3 haplotype comprises one or more markers that fall within the interval flanked by and including physical position 4157916-7054678 on LG-J on the Glyma1 soybean genome assembly.

In further examples, the at least one Rag haplotype comprises marker loci selected from the group consisting of: (a) one or more marker loci selected from the group consisting of S14181-1-Q1, S13871-1-Q1, S14161-1-Q10, S09515-1-Q1, S14151-1-Q1, S14151-2-Q4, S07164-1-Q12, S14182-1-Q1, S00812-1-A, and S02780-1-A; (b) one or more marker loci selected from the group consisting of S01190-1-A, S14761-001-Q001, S14771-001-Q001, S07165-1-Q3, S14778-00′-Q001, and S01164-1-Q1; and (c) one or more marker loci selected from the group consisting of S13662-1-Q3/Q6, S13663-1-Q1, S11411-1-Q1, S13664-1-Q1/Q002, S13672-1-Q1/Q2/Q3, S13674-1-Q1/Q007, and S13675-2-Q1. In still further examples, the at least one Rag haplotype detected comprises two or more of the marker loci within one or more of (a), (b), or (c). In other examples, the at least one Rag haplotype detected comprises three or more of the marker loci within one or more of (a), (b), or (c). In yet other examples, the at least one Rag haplotype detected comprises four or more of the marker loci within one or more of (a), (b), or (c). In even further examples, the at least one Rag haplotype detected comprises all of the marker loci within one or more of (a), (b), or (c).

In still further examples, the method comprises detecting a marker profile comprising two or more of the Rag haplotypes of (a), (b), and (c). In even further examples, the method comprises detecting a marker profile comprising all three of the Rag haplotypes of (a), (b), and (c).

In some examples, the detecting comprises amplifying at least one of said marker loci or a portion thereof and detecting the resulting amplified marker amplicon. In certain examples, the amplifying comprises: (a) admixing an amplification primer or amplification primer pair with a nucleic acid isolated from the first soybean plant or germplasm, wherein the primer or primer pair is complementary or partially complementary to at least a portion of the marker locus, and is capable of initiating DNA polymerization by a DNA polymerase using the soybean nucleic acid as a template; and, (b) extending the primer or primer pair in a DNA polymerization reaction comprising a DNA polymerase and a template nucleic acid to generate at least one amplicon. In some examples, the method employs the target regions and/or primers provided in Table 1. In some particular examples, the method comprises amplifying at least a portion of one or more genome regions selected from the group consisting of SEQ ID NOs: 5, 10, 15, 20, 25, 30, 35, 40, 45, 50, 55, 60, 65, 70, 75, 84, 89, 94, 103, 114, 121, 126, and 131. In other examples, the primer or primer pair comprises a nucleic acid sequence selected from the group consisting of SEQ ID NOs: 1, 2, 6, 7, 11, 12, 16, 17, 21, 22, 26, 27, 31, 32, 36, 37, 41, 42, 46, 47, 51, 52, 56, 57, 61, 62, 66, 67, 71, 72, 76, 77, 80, 81, 85, 86, 90, 91, 95, 96, 99, 100, 104, 105, 108, 109, 110, 111, 115, 116, 119, 120, 122, 123, 127, and 128.

In certain other examples, the detecting further comprises providing a detectable probe. In certain examples, the probes used for detection are those provided in Table 1. In some particular examples, the probe comprises a nucleic acid sequence selected from the group consisting of SEQ ID NOs: 3, 4, 8, 9, 13, 14, 18, 19, 23, 24, 28, 29, 33, 34, 38, 39, 43, 44, 48, 49, 53, 54, 58, 59, 63, 64, 68, 69, 73, 74, 78, 79, 82, 83, 87, 88, 92, 93, 97, 98, 101, 102, 106, 107, 112, 113, 117, 118, 124, 125, 129, and 130. In other examples, the probe comprises at least a portion of a nucleic acid sequence selected from the group consisting of SEQ ID NOs: 5, 10, 15, 20, 25, 30, 35 40, 45, 50, 55, 60, 65, 70, 75, 84, 89, 94, 103, 114, 121, 126, and 131.

In still further aspects, the information disclosed herein regarding haplotypes and marker profiles related to resistance to soybean aphid can be used to aid in the selection of breeding plants, lines, and populations containing improved resistance to soybean aphid for use in introgression of this trait into elite soybean germplasm, or germplasm of proven genetic superiority suitable for variety release. Also provided is a method for introgressing a soybean QTL, haplotype, or marker profile associated with soybean aphid resistance into non-resistant soybean germplasm or less resistant soybean germplasm. According to the method, haplotypes and/or marker profiles are used to select soybean plants containing the improved resistance trait. Plants so selected can be used in a soybean breeding program. Through the process of introgression, the QTL, haplotype, or marker profile associated with improved soybean aphid resistance is introduced from plants identified using marker-assisted selection (MAS) to other plants. According to the method, agronomically desirable plants and seeds can be produced containing the QTL, haplotype, or marker profile associated with soybean aphid resistance from germplasm containing the QTL, haplotype, or marker profile. Sources of improved soybean aphid resistance are disclosed below.

Also provided herein is a method for producing a soybean plant adapted for conferring improved soybean aphid resistance. First, donor soybean plants for a parental line containing the aphid resistance QTL, haplotype, and/or marker profile are selected. In certain examples, the donor soybean plant or germplasm comprises a soybean variety selected from the group consisting of PI567666, PI567622, PI219652, PI219655, 95B97, PI587577E, PI587973B, PI567392, PI567055, PI567063, FC031416, PI507089B, and PI567183. In other examples, the donor soybean plant or germplasm comprises a soybean variety selected from the group consisting of PI567666, PI567622, and PI507089E. In further examples, the donor soybean plant or germplasm comprises a soybean variety selected from the group consisting of PI587577E, PI587973B, PI567392, and PI567183. In additional examples, the donor soybean plant or germplasm comprises a soybean variety selected from the group consisting of PI219652, PI219655, PI567063, and FC031416. In yet further examples, the donor soybean plant or germplasm comprises soybean variety PI567055. In other examples, the donor soybean plant or germplasm comprises a soybean variety selected from the group consisting of PI567666, PI567622, PI219652, and PI219655. According to the method, selection can be accomplished via MAS as explained herein. Selected plant material may represent, among others, an inbred line, a hybrid line, a heterogeneous population of soybean plants, or an individual plant. According to techniques well known in the art of plant breeding, this donor parental line is crossed with a second parental line. In some examples, the second parental line is a high yielding line. This cross produces a segregating plant population composed of genetically heterogeneous plants. Plants of the segregating plant population are screened for the soybean aphid resistance QTL, haplotype, or marker profile. Further breeding may include, among other techniques, additional crosses with other lines, hybrids, backcrossing, or self-crossing. The result is a line of soybean plants that has improved resistance to soybean aphid and optionally also has other desirable traits from one or more other soybean lines.

Plants, including soybean plants, seeds, tissue cultures, variants and mutants, having improved soybean aphid resistance are also provided. In certain examples, plants produced by the foregoing methods are provided. In other examples, plants comprising the Rag haplotypes or marker profiles discussed herein are provided. In yet further examples, plants comprising favorable or disfavored alleles at the marker loci discussed herein are provided. In certain examples, plants comprising a Rag haplotype selected from the group consisting of Rag1-b, Rag1-c, Rag2-d, Rag3-b, and Rag3-d are provided. In certain other examples, plants comprising a haplotype or marker profile selected from the group consisting of (a) Rag1-b/Rag3-b; (b) Rag1-b; (c) Rag1-c/Rag3-d; (d) Rag1-e; and (e) Rag1-d/Rag2-c are provided. In yet further examples, plants comprising a favorable or disfavored allele at (a) one or more marker loci selected from the group consisting S14181-1-Q1, S13871-1-Q1, S14161-1-Q10, S09515-1-Q1, S14151-1-Q1, S14151-2-Q4, S07164-1-Q12, S14182-1-Q1, S00812-1-A, and S02780-1-A; (b) one or more marker loci selected from the group consisting of S01190-1-A, S14761-001-Q001, S14771-001-Q001, S07165-1-Q3, S14778-001-Q001, and S01164-1-Q1; or (c) one or more marker loci selected from the group consisting of S13662-1-Q3/Q6, S13663-1-Q1, S11411-1-Q1, S13664-1-Q1/Q002, S13672-1-Q1/Q2/Q3, S13674-1-Q1/Q007, and S13675-2-Q1 are provided. In further examples, plants comprising a favorable or disfavored allele at (a) the marker loci S14161-1-Q10, S09515-1-Q1, S14151-2-Q4, and S07164-1-Q12; (b) the marker loci S07165-1-Q3, S01190-1-A, and S01164-1-Q1; or (c) the marker loci S11411-1-Q1, S13674-1-Q1/Q007, and S13675-2-Q1 are provided.

Also provided are isolated nucleic acids, kits, and systems useful for the identification and selection methods disclosed herein. In certain examples, isolated nucleic acids, kits, and systems useful for the detection of the Rag haplotypes or marker profiles discussed herein are provided. In yet further examples, isolated nucleic acids, kits, and systems useful for the detection of the favorable or disfavored alleles at the marker loci discussed herein are provided. In certain examples, isolated nucleic acids, kits, and systems useful for the detection of a Rag haplotype selected from the group consisting of Rag1-b, Rag1-c, Rag2-d, Rag3-b, and Rag3-d are provided. In certain other examples, isolated nucleic acids, kits, and systems useful for the detection of a haplotype or marker profile selected from the group consisting of (a) Rag1-b/Rag3-b; (b) Rag1-b; (c) Rag1-c/Rag3-d; (d) Rag 1-e; and (e) Rag1-d/Rag2-c are provided. In yet further examples, isolated nucleic acids, kits, and systems useful for the detection of a favorable or disfavored allele at (a) one or more marker loci selected from the group consisting S14181-1-Q1, S13871-1-Q1, S14161-1-Q10, S09515-1-Q1, S14151-1-Q1, S14151-2-Q4, S07164-1-Q12, S14182-1-Q1, S00812-1-A, and S02780-1-A; (b) one or more marker loci selected from the group consisting of S01190-1-A, S14761-001-Q001, S14771-001-Q001, S07165-1-Q3, S14778-001-Q001, and S01164-1-Q1; or (c) one or more marker loci selected from the group consisting of S13662-1-Q3/Q6, S13663-1-Q1, S11411-1-Q1, S13664-1-Q1/Q002, S13672-1-Q1/Q2/Q3, S13674-1-Q1/Q007, and S13675-2-Q1 are provided. In further examples, isolated nucleic acids, kits, and systems useful for the detection of a favorable or disfavored allele at (a) the marker loci S14161-1-Q10, S09515-1-Q1, S14151-2-Q4, and S07164-1-Q12; (b) the marker loci S07165-1-Q3, 501190-1-A, and S01164-1-Q1; or (c) the marker loci S11411-1-Q1, S13674-1-Q1/Q007, and S13675-2-Q1 are provided.

It is to be understood that this invention is not limited to particular embodiments, which can, of course, vary. It is also to be understood that the terminology used herein is for the purpose of describing particular embodiments only, and is not intended to be limiting. Further, all publications referred to herein are incorporated by reference herein for the purpose cited to the same extent as if each was specifically and individually indicated to be incorporated by reference herein.

### DEFINITIONS

As used in this specification and the appended claims, terms in the singular and the singular forms “a,” “an,” and “the,” for example, include plural referents unless the content clearly dictates otherwise. Thus, for example, reference to “plant,” “the plant,” or “a plant” also includes a plurality of plants; also, depending on the context, use of the term “plant” can also include genetically similar or identical progeny of that plant; use of the term “a nucleic acid” optionally includes, as a practical matter, many copies of that nucleic acid molecule; similarly, the term “probe” optionally (and typically) encompasses many similar or identical probe molecules.

Additionally, as used herein, “comprising” is to be interpreted as specifying the presence of the stated features, integers, steps, or components as referred to, but does not preclude the presence or addition of one or more features, integers, steps, or components, or groups thereof. Thus, for example, a kit comprising one pair of oligonucleotide primers may have two or more pairs of oligonucleotide primers. Additionally, the term “comprising” is intended to include examples encompassed by the terms “consisting essentially of” and “consisting of:” Similarly, the term “consisting essentially of” is intended to include examples encompassed by the term “consisting of”

Certain definitions used in the specification and claims are provided below. In order to provide a clear and consistent understanding of the specification and claims, including the scope to be given such terms, the following definitions are provided:

“Agronomics,” “agronomic traits,” and “agronomic performance” refer to the traits (and underlying genetic elements) of a given plant variety that contribute to yield over the course of a growing season. Individual agronomic traits include emergence vigor, vegetative vigor, stress tolerance, disease resistance or tolerance, insect resistance or tolerance, herbicide resistance, branching, flowering, seed set, seed size, seed density, standability, threshability, and the like.

“Allele” means any of one or more alternative forms of a genetic sequence. In a diploid cell or organism, the two alleles of a given sequence typically occupy corresponding loci on a pair of homologous chromosomes. With regard to a SNP marker, allele refers to the specific nucleotide base present at that SNP locus in that individual plant.

The term “amplifying” in the context of nucleic acid amplification is any process whereby additional copies of a selected nucleic acid (or a transcribed form thereof) are produced. Typical amplification methods include various polymerase based replication methods, including the polymerase chain reaction (PCR), ligase mediated methods, such as the ligase chain reaction (LCR), and RNA polymerase based amplification (e.g., by transcription) methods. An “amplicon” is an amplified nucleic acid, e.g., a nucleic acid that is produced by amplifying a template nucleic acid by any available amplification method (e.g., PCR, LCR, transcription, or the like).

An “ancestral line” is a parent line used as a source of genes, e.g., for the development of elite lines.

An “ancestral population” is a group of ancestors that have contributed the bulk of the genetic variation that was used to develop elite lines.

“Backcrossing” is a process in which a breeder crosses a progeny variety back to one of the parental genotypes one or more times.

“Biotype” or “aphid biotype” means a subspecies of soybean aphid that share certain genetic traits or a specified genotype. There are currently three well-documented biotypes of soybean aphid: Urbana, Ill. (biotype 1), Wooster, Ohio (biotype 2), and Indiana (biotype 3). An additional biotype, referred to herein as biotype X, was collected from soybean fields in Lime Springs, Iowa.

The term “chromosome segment” designates a contiguous linear span of genomic DNA that resides in planta on a single chromosome.

“Cultivar” and “variety” are used synonymously and mean a group of plants within a species (e.g., Glycine max) that share certain genetic traits that separate them from other possible varieties within that species. Soybean cultivars are inbred lines produced after several generations of self-pollinations. Individuals within a soybean cultivar are homogeneous, nearly genetically identical, with most loci in the homozygous state.

An “elite line” is an agronomically superior line that has resulted from many cycles of breeding and selection for superior agronomic performance. Numerous elite lines are available and known to those of skill in the art of soybean breeding.

An “elite population” is an assortment of elite individuals or lines that can be used to represent the state of the art in terms of agronomically superior genotypes of a given crop species, such as soybean.

An “exotic soybean strain” or an “exotic soybean germplasm” is a strain or germplasm derived from a soybean not belonging to an available elite soybean line or strain of germplasm. In the context of a cross between two soybean plants or strains of germplasm, an exotic germplasm is not closely related by descent to the elite germplasm with which it is crossed. Most commonly, the exotic germplasm is not derived from any known elite line of soybean, but rather is selected to introduce novel genetic elements (typically novel alleles) into a breeding program.

A “genetic map” is a description of genetic linkage relationships among loci on one or more chromosomes (or linkage groups) within a given species, generally depicted in a diagrammatic or tabular form.

“Genotype” refers to the genetic constitution of a cell or organism.

“Germplasm” means the genetic material that comprises the physical foundation of the hereditary qualities of an organism. As used herein, germplasm includes seeds and living tissue from which new plants may be grown; or, another plant part, such as leaf, stem, pollen, or cells, that may be cultured into a whole plant. Germplasm resources provide sources of genetic traits used by plant breeders to improve commercial cultivars.

An individual is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

“Intro gression” means the entry or introduction of a gene, QTL, haplotype, marker profile, trait, or trait locus from the genome of one plant into the genome of another plant.

A “line” or “strain” is a group of individuals of identical parentage that are generally inbred to some degree and that are generally homozygous and homogeneous at most loci (isogenic or near isogenic). A “subline” refers to an inbred subset of descendents that are genetically distinct from other similarly inbred subsets descended from the same progenitor. Traditionally, a subline has been derived by inbreeding the seed from an individual soybean plant selected at the F3 to F5 generation until the residual segregating loci are “fixed” or homozygous across most or all loci. Commercial soybean varieties (or lines) are typically produced by aggregating (“bulking”) the self-pollinated progeny of a single F3 to F5 plant from a controlled cross between 2 genetically different parents. While the variety typically appears uniform, the self-pollinating variety derived from the selected plant eventually (e.g., F8) becomes a mixture of homozygous plants that can vary in genotype at any locus that was heterozygous in the originally selected F3 to F5 plant. Marker-based sublines that differ from each other based on qualitative polymorphism at the DNA level at one or more specific marker loci are derived by genotyping a sample of seed derived from individual self-pollinated progeny derived from a selected F3-F5 plant. The seed sample can be genotyped directly as seed, or as plant tissue grown from such a seed sample. Optionally, seed sharing a common genotype at the specified locus (or loci) are bulked providing a subline that is genetically homogenous at identified loci important for a trait of interest (e.g., yield, tolerance, etc.).

“Linkage” refers to a phenomenon wherein alleles on the same chromosome tend to segregate together more often than expected by chance if their transmission was independent. Genetic recombination occurs with an assumed random frequency over the entire genome. Genetic maps are constructed by measuring the frequency of recombination between pairs of traits or markers. The closer the traits or markers are to each other on the chromosome, the lower the frequency of recombination, and the greater the degree of linkage. Traits or markers are considered herein to be linked if they generally co-segregate. A 1/100 probability of recombination per generation is defined as a map distance of 1.0 centiMorgan (1.0 cM).

The genetic elements or genes located on a single chromosome segment are physically linked. Advantageously, the two loci are located in close proximity such that recombination between homologous chromosome pairs does not occur between the two loci during meiosis with high frequency, e.g., such that linked loci co-segregate at least about 90% of the time, e.g., 91%, 92%, 93%, 94%, 95%, 96%, 97%, 98%, 99%, 99.5%, 99.75%, or more of the time. The genetic elements located within a chromosome segment are also genetically linked, typically within a genetic recombination distance of less than or equal to 50 centimorgans (cM), e.g., about 49, 40, 30, 20, 10, 5, 4, 3, 2, 1, 0.75, 0.5, or 0.25 cM or less. That is, two genetic elements within a single chromosome segment undergo recombination during meiosis with each other at a frequency of less than or equal to about 50%, e.g., about 49%, 40%, 30%, 20%, 10%, 5%, 4%, 3%, 2%, 1%, 0.75%, 0.5%, or 0.25% or less. Closely linked markers display a cross over frequency with a given marker of about 10% or less (the given marker is within about 10cM of a closely linked marker). Put another way, closely linked loci co-segregate at least about 90% of the time.

When referring to the relationship between two genetic elements, such as a genetic element contributing to resistance and a proximal marker, “coupling” phase linkage indicates the state where the “favorable” allele at the resistance locus is physically associated on the same chromosome strand as the “favorable” allele of the respective linked marker locus. In coupling phase, both favorable alleles are inherited together by progeny that inherit that chromosome strand. In “repulsion” phase linkage, the “favorable” allele at the locus of interest (e.g., a QTL for resistance) is physically linked with an “unfavorable” allele at the proximal marker locus, and the two “favorable” alleles are not inherited together (i.e., the two loci are “out of phase” with each other).

“Linkage disequilibrium” refers to a phenomenon wherein alleles tend to remain together in linkage groups when segregating from parents to offspring, with a greater frequency than expected from their individual frequencies.

“Linkage group” (LG) refers to traits or markers that generally co-segregate. A linkage group generally corresponds to a chromosomal region containing genetic material that encodes the traits or markers. As such, a linkage group can generally be assigned to a certain chromosome, and such associations are well known in the art, for example from the soybase database (soybase.org). For example, soybean LG-M corresponds to soybean chromosome 7, soybean LG-F corresponds to soybean chromosome 13, and soybean LG-J corresponds to soybean chromosome 16.

“Locus” is a defined segment of DNA.

A “map location” is an assigned location on a genetic map relative to linked genetic markers where a specified marker can be found within a given species.

“Mapping” is the process of defining the linkage relationships of loci through the use of genetic markers, populations segregating for the markers, and standard genetic principles of recombination frequency.

“Marker” or “molecular marker” is a term used to denote a nucleic acid or amino acid sequence that is sufficiently unique to characterize a specific locus on the genome. Examples include Restriction Fragment Length Polymorphisms (RFLPs), Single Sequence Repeats (SSRs), Target Region Amplification Polymorphisms (TRAPs), Isozyme Electrophoresis, Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), and Single Nucleotide Polymorphisms (SNPs). Additionally, other types of molecular markers are known in the art, and phenotypic traits may also be used as markers in the methods. All markers are used to define a specific locus on the soybean genome. Large numbers of these markers have been mapped (see, e.g., the Soybase database at soybase.org). Each marker is therefore an indicator of a specific segment of DNA, having a unique nucleotide sequence. The map positions provide a measure of the relative positions of particular markers with respect to one another. When a trait is stated to be linked to a given marker it will be understood that the actual DNA segment whose sequence affects the trait generally co-segregates with the marker. More precise and definite localization of a trait can be obtained if markers are identified on both sides of the trait. By measuring the appearance of the marker(s) in progeny of crosses, the existence of the trait can be detected by relatively simple molecular tests without actually evaluating the appearance of the trait itself, which can be difficult and time-consuming because the actual evaluation of the trait requires growing plants to a stage where the trait can be expressed. Molecular markers have been widely used to determine genetic composition in soybeans.

“Marker assisted selection” refers to the process of selecting a desired trait or traits in a plant or plants by detecting one or more nucleic acids from the plant, where the nucleic acid is linked to the desired trait, and then selecting the plant or germplasm possessing those one or more nucleic acids.

The term “plant” includes reference to an immature or mature whole plant, including a plant from which seed or grain or anthers have been removed. Seed or embryo that will produce the plant is also considered to be the plant.

“Plant parts” means any portion or piece of a plant, including leaves, stems, buds, roots, root tips, anthers, seed, grain, embryo, pollen, ovules, flowers, cotyledons, hypocotyls, pods, flowers, shoots, stalks, tissues, tissue cultures, cells and the like.

“Polymorphism” means a change or difference between two related nucleic acids. A “nucleotide polymorphism” refers to a nucleotide that is different in one sequence when compared to a related sequence when the two nucleic acids are aligned for maximal correspondence.

“Polynucleotide,” “polynucleotide sequence,” “nucleic acid sequence,” “nucleic acid fragment,” and “oligonucleotide” are used interchangeably herein. These terms encompass nucleotide sequences and the like. A polynucleotide may be a polymer of RNA or DNA that is single- or double-stranded, that optionally contains synthetic, non-natural, or altered nucleotide bases. A polynucleotide in the form of a polymer of DNA may be comprised of one or more strands of cDNA, genomic DNA, synthetic DNA, or mixtures thereof.

“Primer” refers to an oligonucleotide (synthetic or occurring naturally), which is capable of acting as a point of initiation of nucleic acid synthesis or replication along a complementary strand when placed under conditions in which synthesis of a complementary strand is catalyzed by a polymerase. Typically, primers are oligonucleotides from 10 to 30 nucleic acids in length, but longer or shorter sequences can be employed. Primers may be provided in double-stranded form, though the single-stranded form is preferred. A primer can further contain a detectable label, for example a 5′ end label.

“Probe” refers to an oligonucleotide (synthetic or occurring naturally) that is complementary (though not necessarily fully complementary) to a polynucleotide of interest and forms a duplexed structure by hybridization with at least one strand of the polynucleotide of interest. Typically, probes are oligonucleotides from 10 to 50 nucleic acids in length, but longer or shorter sequences can be employed. A probe can further contain a detectable label. The terms “label” and “detectable label” refer to a molecule capable of detection, including, but not limited to, radioactive isotopes, fluorescers, chemiluminescers, enzymes, enzyme substrates, enzyme cofactors, enzyme inhibitors, chromophores, dyes, metal ions, metal sols, semiconductor nanocrystals, ligands (e.g., biotin, avidin, streptavidin, or haptens), and the like. A detectable label can also include a combination of a reporter and a quencher, such as are employed in FRET probes or TaqMan™ probes. The term “reporter” refers to a substance or a portion thereof which is capable of exhibiting a detectable signal, which signal can be suppressed by a quencher. The detectable signal of the reporter is, e.g., fluorescence in the detectable range. The term “quencher” refers to a substance or portion thereof which is capable of suppressing, reducing, inhibiting, etc., the detectable signal produced by the reporter. As used herein, the terms “quenching” and “fluorescence energy transfer” refer to the process whereby, when a reporter and a quencher are in close proximity, and the reporter is excited by an energy source, a substantial portion of the energy of the excited state nonradiatively transfers to the quencher where it either dissipates nonradiatively or is emitted at a different emission wavelength than that of the reporter.

“PRMMAT” means Predicted Relative Maturity. Soybean maturities are divided into relative maturity groups. In the United States the most common maturity groups are 00 through VIII. Within maturity groups 00 through V are sub-groups. A sub-group is a tenth of a relative maturity group. Within narrow comparisons, the difference of a tenth of a relative maturity group equates very roughly to a day difference in maturity at harvest.

“Rag genes,” “Rag intervals,” “Rag QTL,” and “Rag loci” refer to one or more of the Rag1, Rag2, and Rag3 genes and the chromosome segments or intervals on which they are located. Rag1 maps to linkage group M. In some examples, the Rag1 interval is defined as being flanked by and including markers Satt540 and BARC-016783-02329. In other examples, the Rag1 interval is defined as being flanked by and including markers BARC-039195-07466 and BARC-016783-02329. Rag2 maps to linkage group F. In some examples, the Rag2 interval is defined as being flanked by and including markers Satt334 and Sat_{—}317. In other examples, the Rag2 interval is defined as being flanked by and including markers BARC-029823-06424 and Sct_{—}033. Rag3 maps to linkage group J. In some examples, the Rag 3 interval is defined as being flanked by and including markers Sat_{—}339 and Sct_{—}065. In other examples, the Rag3 interval is defined as being flanked by and including markers BARC-031195-07010 and Sat_{—}370.

“Rag haplotype” or simply “haplotype” means the combination of particular alleles present within a particular plant's genome at one or more specific marker loci within or linked to the Rag1, Rag2, or Rag3 interval or gene. For instance, in one example, one specific marker locus within or linked to the Rag1 interval is used to define a Rag1 haplotype for a particular plant. In another example, two specific marker loci within or linked to the Rag1 interval are used to define a Rag1 haplotype for a particular plant. In still further examples, 3, 4, 5, 6, 7, 8, 9, 10, 11, 12, 13, 14, 15, 16, 17, 18, 19, 20, or more specific marker loci within or linked to the Rag1 interval are used to define a Rag1 haplotype for a particular plant. The same applies for the Rag2 and Rag3 intervals.

In certain examples, multiple Rag haplotypes are used to define a “marker profile” or “Rag marker profile.” As used herein, “marker profile” means the combination of two or more Rag haplotypes within a particular plant's genome. For instance, in one example, a particular Rag1 haplotype and a particular Rag2 haplotype define the marker profile of a particular plant. In another example, a particular Rag1 haplotype and a particular Rag3 haplotype define the marker profile of a particular plant. In a still further example, a particular Rag2 haplotype and a particular Rag3 haplotype define the marker profile of a particular plant. In an additional example, a particular Rag1 haplotype, a particular Rag2 haplotype, and a particular Rag3 haplotype define the marker profile of a particular plant. More specifically, a particular plant marker profile might be, for example, Rag1-a/Rag2-a or Rag1-b/Rag2-a/Rag3-c.

“Recombination frequency” is the frequency of a crossing over event (recombination) between two genetic loci. Recombination frequency can be observed by following the segregation of markers and/or traits during meiosis.

“Resistance” and “improved resistance” are used interchangeably herein and refer to one or more of antibiosis resistance, antixenosis resistance, and tolerance to soybean aphid. “Antibiosis” refers to the plant's ability to reduce the survival, reproduction, and fecundity of the insect. “Antixenosis” refers to the plant's ability to deter the insect from feeding or identifying the plant as a food source. “Tolerance” refers to the plant's ability to withstand heavy infestation without significant yield loss. A “resistant plant” or “resistant plant variety” need not possess absolute or complete resistance to one or more soybean aphid biotypes. Instead, a “resistant plant,” “resistant plant variety,” or a plant or plant variety with “improved resistance” will have a level of resistance to at least one soybean aphid biotype which is higher than that of a comparable susceptible plant or variety.

“Self crossing,” “self pollination,” or “selfing” is a process through which a breeder crosses a plant with itself; for example, a second generation hybrid F2 with itself to yield progeny designated F2:3.

“SNP” or “single nucleotide polymorphism” means a sequence variation that occurs when a single nucleotide (A, T, C, or G) in the genome sequence is altered or variable. “SNP markers” exist when SNPs are mapped to sites on the soybean genome. Many techniques for detecting SNPs are known in the art, including allele specific hybridization, primer extension, direct sequencing, and real-time PCR, such as the TaqMan™ assay.

“Transgenic plant” refers to a plant that comprises within its cells an exogenous polynucleotide. Generally, the exogenous polynucleotide is stably integrated within the genome such that the polynucleotide is passed on to successive generations. The exogenous polynucleotide may be integrated into the genome alone or as part of a recombinant expression cassette. “Transgenic” is used herein to refer to any cell, cell line, callus, tissue, plant part, or plant, the genotype of which has been altered by the presence of exogenous nucleic acid including those transgenic organisms or cells initially so altered, as well as those created by crosses or asexual propagation from the initial transgenic organism or cell. The term “transgenic” as used herein does not encompass the alteration of the genome (chromosomal or extra-chromosomal) by conventional plant breeding methods (e.g., crosses) or by naturally occurring events such as random cross-fertilization, non-recombinant viral infection, non-recombinant bacterial transformation, non-recombinant transposition, or spontaneous mutation.

The term “vector” is used in reference to polynucleotide or other molecules that transfer nucleic acid segment(s) into a cell. A vector optionally comprises parts which mediate vector maintenance and enable its intended use (e.g., sequences necessary for replication, genes imparting drug or antibiotic resistance, a multiple cloning site, operably linked promoter/enhancer elements which enable the expression of a cloned gene, etc.). Vectors are often derived from plasmids, bacteriophages, or plant or animal viruses.

The term “yield” refers to the productivity per unit area of a particular plant product of commercial value. For example, yield of soybean is commonly measured in bushels of seed per acre or metric tons of seed per hectare per season. Yield is affected by both genetic and environmental factors. Yield is the final culmination of all agronomic traits.

SNP Markers, Rag Haplotypes, and Marker Profiles Associated with Resistance to Soybean Aphid:

Markers, primers, haplotypes, and marker profiles, and methods of their use for identifying and/or selecting soybean plants with improved soybean aphid resistance, are provided. The method for determining the presence/absence/allele of a particular marker associated with soybean aphid resistance and within or linked to a Rag gene or interval in soybean plant or germplasm, and in turn determining the Rag haplotype and/or marker profile of the plant/germplasm, comprises analyzing genomic DNA from a soybean plant or germplasm to determine if at least one, or a plurality, of such markers is present or absent and in what allelic form. Using this information regarding the Rag-associated markers present in the particular plant or germplasm in turn allows a Rag haplotype to be assigned to that plant/germplasm. If multiple Rag haplotypes are deduced for a plant, a marker profile can in turn be assigned by combining all of these Rag haplotypes.

In certain examples, plants or germplasm are identified that have at least one favorable allele, haplotype, or marker profile that positively correlates with resistance or improved resistance. However, in other examples, it is useful for exclusionary purposes during breeding to identify alleles, haplotypes, or marker profiles that negatively correlate with resistance, for example to eliminate such plants or germplasm from subsequent rounds of breeding.

While any marker linked to a Rag gene or interval is useful, markers that map closer to a Rag gene or interval are generally preferred over markers that map farther from a Rag gene or interval. Marker loci are especially useful when they are closely linked to a Rag gene or interval. Thus, in one example, marker loci display an inter-locus cross-over frequency of about 10% or less, about 9% or less, about 8% or less, about 7% or less, about 6% or less, about 5% or less, about 4% or less, about 3% or less, about 2% or less, about 1% or less, about 0.75% or less, about 0.5% or less, or about 0.25% or less with the Rag gene to which they are linked. Thus, the loci are separated from the Rag gene to which they are linked by about 10 cM, 9 cM, 8 cM, 7 cM, 6 cM, 5 cM, 4 cM, 3 cM, 2 cM, 1 cM, 0.75 cM, 0.5 cM, or 0.25 cM or less.

In certain examples, multiple marker loci that collectively make up the Rag haplotype of interest are investigated, for instance 2, 3, 4, 5, 6, 7, 8, 9, 10, 11, 12, 13, 14, 15, 16, 17, 18, 19, 20, 21, 22, 23, 24, 25, 26, 27, 28, 29, 30, or more marker loci.

In certain examples, markers useful for defining a Rag1 haplotype are linked or are closely linked to the interval flanked by and including the marker loci Satt540 and BARC-016783-02329 in the Soybase database (soybase.org). In other examples, markers useful for defining a Rag1 haplotype are linked or are closely linked to the interval flanked by and including the marker loci BARC-039195-07466 and BARC-016783-02329 in the Soybase database (soybase.org). In still further examples, markers useful for defining a Rag1 haplotype are within the interval flanked by and including Satt540 and BARC-016783-02329 or BARC-039195-07466 and BARC-016783-02329 in the Soybase database (soybase.org). In other particular examples, the markers useful for defining a Rag1 haplotype are within the interval flanked by and including Satt435 and Sat_{—}244 in the Soybase database (soybase.org). In further particular examples, the markers useful for defining a Rag1 haplotype are within the interval flanked by and including physical position 5464314-8194502 on LG-M on the Glyma1 soybean genome assembly.

In additional examples, markers useful for defining a Rag2 haplotype are linked or are closely linked to the interval flanked by and including the marker loci Satt334 and Sat_{—}317 in the Soybase database (soybase.org). In other examples, markers useful for defining a Rag2 haplotype are linked to or are closely linked to the interval flanked by and including the marker loci BARC-029823-06424 and Sct_{—}033 in the Soybase database (soybase.org). In still further examples, markers useful for defining a Rag2 haplotype are within the interval flanked by and including Satt334 and Sat_{—}317 or BARC-029823-06424 and Sct_{—}033 in the Soybase database (soybase.org). In other particular examples, the markers useful for defining a Rag2 haplotype are within the interval flanked by and including Satt334 and Satt510 in the Soybase database (soybase.org). In further particular examples, the markers useful for defining a Rag2 haplotype are within the interval flanked by and including physical position 28416122-30590233 on LG-F on the Glyma1 soybean genome assembly.

In yet further examples, markers useful for defining a Rag3 haplotype are linked or are closely linked to the interval flanked by and including the marker loci Sat_{—}339 and Sct_{—}065 in the Soybase database (soybase.org). In still further examples, markers useful for defining a Rag3 haplotype are linked or are closely linked to the interval flanked by and including the marker loci BARC-031195-07010 and Sat_{—}370 in the Soybase database (soybase.org). In still further examples, markers useful for defining a Rag3 haplotype are within the interval flanked by and including Sat_{—}339 and Sct_{—}065 or BARC-031195-07010 and Sat_{—}370 in the Soybase database (soybase.org). In other particular examples, the markers useful for defining a Rag3 haplotype are within the interval flanked by and including Sat_{—}339 and Sat_{—}370 in the Soybase database (soybase.org). In further particular examples, the markers useful for defining a Rag3 haplotype are within the interval flanked by and including physical position 4157916-7054678 on LG-J on the Glyma1 soybean genome assembly.

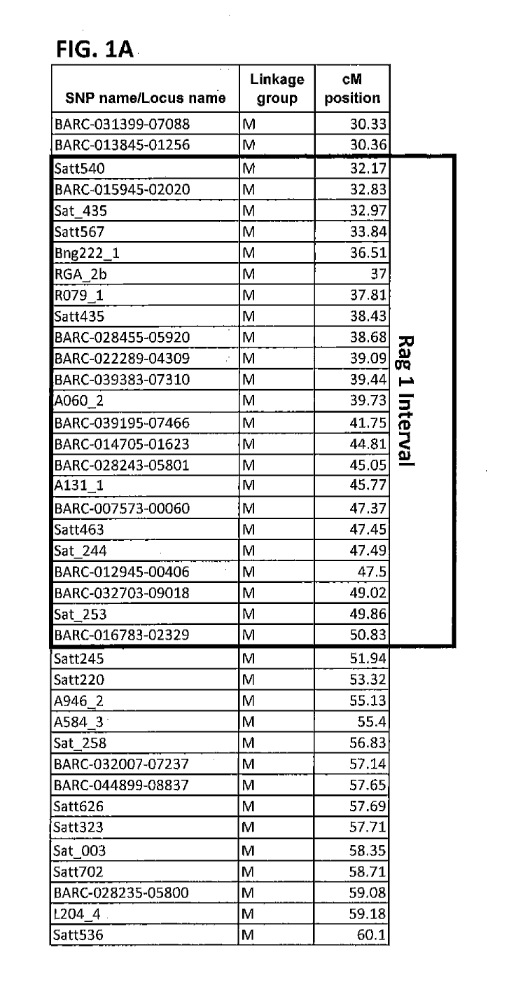

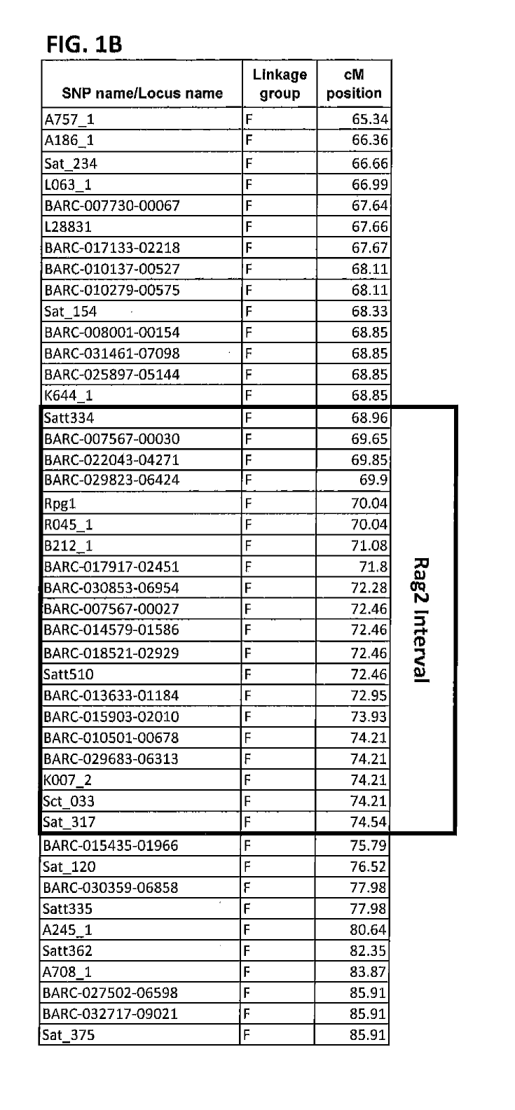

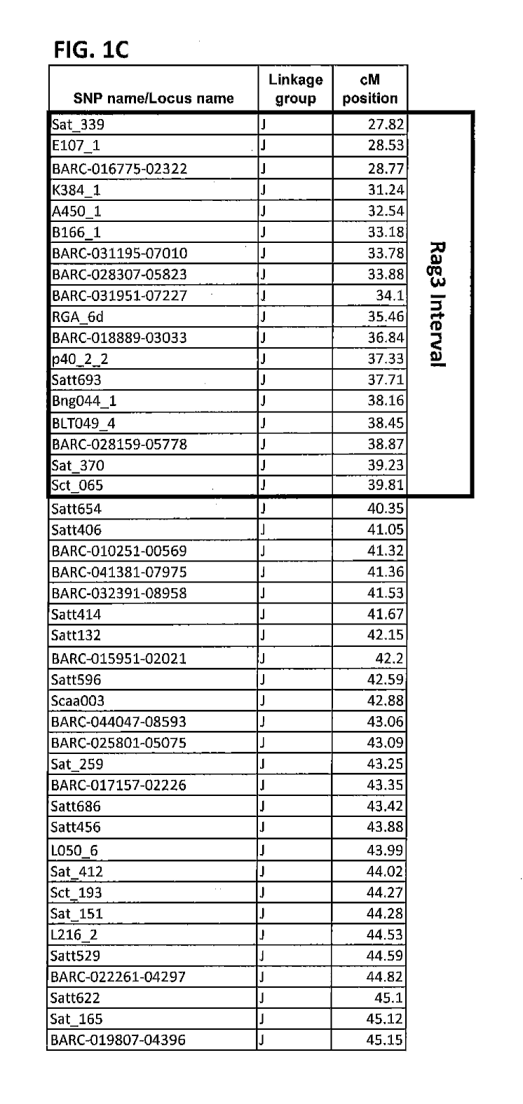

Markers within, linked to, or closely linked to these intervals are illustrated in the genetic map of FIG. 1. Numerous such markers are also well known in the art, for example, are described in the USDA's soybase database, available at www.soybase.org.

Exemplary markers useful for defining Rag haplotypes are provided in Table 1. Also provided in Table 1 are the target regions containing the markers, as well as primers and probes that can be used to amplify and detect the markers.

In certain examples the marker loci used to define the Rag1 haplotype are one or more of S14181-1-Q1, S13871-1-Q1, S14161-1-Q10, S09515-1-Q1, S14151-1-Q1, S14151-2-Q4, S07164-1-Q12, S14182-1-Q1, S00812-1-A, and S02780-1-A. In other examples, the marker loci used to define the Rag1 haplotype are two or more of S14181-1-Q1, S13871-1-Q1, S14161-1-Q10, S09515-1-Q1, S14151-1-Q1, S14151-2-Q4, S07164-1-Q12, S14182-1-Q1, S00812-1-A, and S02780-1-A. In further examples, the marker loci used to define the Rag1 haplotype are three or more of S14181-1-Q1, S13871-1-Q1, S14161-1-Q10, S09515-1-Q1, S14151-1-Q1, S14151-2-Q4, S07164-1-Q12, S14182-1-Q1, S00812-1-A, and S02780-1-A. In additional examples, the marker loci used to define the Rag1 haplotype are four or more of S14181-1-Q1, S13871-1-Q1, S14161-1-Q10, S09515-1-Q1, S14151-1-Q1, S14151-2-Q4, S07164-1-Q12, S14182-1-Q1, S00812-1-A, and S02780-1-A. In still further examples, the marker loci used to define the Rag1 haplotype are five or more of S14181-1-Q1, S13871-1-Q1, S14161-1-Q10, S09515-1-Q1, S14151-1-Q1, S14151-2-Q4, S07164-1-Q12, S14182-1-Q1, S00812-1-A, and S02780-1-A. In yet further examples, the marker loci used to define the Rag1 haplotype are all of S14181-1-Q1, S13871-1-Q1, S14161-1-Q10, S09515-1-Q1, S14151-1-Q1, S14151-2-Q4, S07164-1-Q12, S14182-1-Q1, S00812-1-A, and S02780-1-A. In a particular example, the marker loci used to define the Rag1 haplotype are all of S14161-1-Q10, S09515-1-Q1, S14151-2-Q4, and S07164-1-Q12.

In certain examples, the marker loci used to define the Rag2 haplotype are one or more of S01190-1-A, S14761-001-Q001, S14771-001-Q001, S07165-1-Q3, S14778-001-Q001, and S01164-1-Q1. In other examples, the marker loci used to define the Rag2 haplotype are two or more of S01190-1-A, S14761-00′-Q001, S14771-001-Q001, S07165-1-Q3, S14778-001-Q001, and S01164-1-Q1. In additional examples, the marker loci used to define the Rag2 haplotype are three or more of S01190-1-A, S14761-001-Q001, S14771-001-Q001, S07165-1-Q3, S14778-00′-Q001, and S01164-1-Q1. In further examples, the marker loci used to define the Rag2 haplotype are four or more of S01190-1-A, S14761-001-Q001, S14771-001-Q001, S07165-1-Q3, S14778-001-Q001, and S01164-1-Q1. In still further examples, the marker loci used to define the Rag2 haplotype are all of S01190-1-A, S14761-00′-Q001, S14771-00′-Q001, S07165-1-Q3, S14778-001-Q001, and S01164-1-Q1. In a particular example, the marker loci used to define the Rag2 haplotype are S01190-1-A, S07165-1-Q3, and S01164-1-Q1.

In certain examples, the marker loci used to define the Rag3 haplotype are one or more of S13662-1-Q3/Q6, S13663-1-Q1, S11411-1-Q1, S13664-1-Q1/Q002, S13672-1-Q1/Q2/Q3, S13674-1-Q1/Q007, and S13675-2-Q1. In other examples, the marker loci used to define the Rag3 haplotype are two or more of S13662-1-Q3/Q6, S13663-1-Q1, S11411-1-Q1, S13664-1-Q1/Q002, S13672-1-Q1/Q2/Q3, S13674-1-Q1/Q007, and S13675-2-Q1. In additional examples, the marker loci used to define the Rag3 haplotype are three or more of S13662-1-Q3/Q6, S13663-1-Q1, S11411-1-Q1, S13664-1-Q1/Q002, S13672-1-Q1/Q2/Q3, S13674-1-Q1/Q007, and S13675-2-Q1. In further examples, the marker loci used to define the Rag3 haplotype are four or more of S13662-1-Q3/Q6, S13663-1-Q1, S11411-1-Q1, S13664-1-Q1/Q002, S13672-1-Q1/Q2/Q3, S13674-1-Q1/Q007, and S13675-2-Q1. In still further examples, the marker loci used to define the Rag3 haplotype are five or more of S13662-1-Q3/Q6, S13663-1-Q1, S11411-1-Q1, S13664-1-Q1/Q002, S13672-1-Q1/Q2/Q3, S13674-1-Q1/Q007, and S13675-2-Q1. examples, the marker loci used to define the Rag3 haplotype are one or more of S13662-1-Q3/Q6, S13663-1-Q1, S11411-1-Q1, S13664-1-Q1/Q002, S13672-1-Q1/Q2/Q3, S13674-1-Q1/Q007, and S13675-2-Q1. In additional examples, the marker loci used to define the Rag3 haplotype are all of S13662-1-Q3/Q6, S13663-1-Q1, S11411-1-Q1, S13664-1-Q1/Q002, S13672-1-Q1/Q2/Q3, S13674-1-Q1/Q007, and S13675-2-Q1. In a particular example, the marker loci used to define the Rag3 haplotype are all of S11411-1-Q1, S13674-1-Q1/Q007, and S13675-2-Q1.

TABLE 1
Selected markers useful for defining Rag haplotypes and marker profiles

| | | | | | |
|---|---|---|---|---|---|
| Composite | | | | | |
| Map | Physical | | | | Probes (probe1-FAM/probe2- |
| Position | pos. of | Geno- | | | VIC; SNP base indicated |
| (cM) | SNP | types | Marker | Forward and Reverse Primers | by capital letter) |

Rag1 (LG-M)

| | | | | | |
|---|---|---|---|---|---|
| 26.06 cM | 5516385 | A/G | S14181- | SEQ ID: 1 gcatctcatgattaagtagg | SEQ ID: 3 caatcaGcacccttg |
| | | | 1-Q1 | SEQ ID: 2 caagaactttgcttgtcttgctg | SEQ ID: 4 aagcaatcaAcaccctt |
| 26.06 cM | 5516818 | C/T | 513871- | SEQ ID: 6 gcaggctcatcagattgctt | SEQ ID: 8 ttgaaacCaccatttt |
| | | | 1-Q1 | SEQ ID: 7 gcagcgtctcatcaacaaaa | SEQ ID: 9 aaacTaccattttgc |
| 26.49 cM | 5598980 | C/G | S14161- | SEQ ID: 11 caccagctcgataagctagagat | SEQ ID: 13 ccagtagcaGcccta |
| | | | 1-Q10 | SEQ ID: 12 ttagccatggattttgttgaatac | SEQ ID: 14 agtagcaCccctaccaa |
| 26.51 cM | 5602544 | A/G | S09515- | SEQ ID: 16 tgcaagattgatttttatgatacgg | SEQ ID: 18 tattgccaAttcgatcc |
| | | | 1-Q1 | SEQ ID: 17 ggactaaaattagaaaaagaggaacca | SEQ ID: 19 tattgccaGttcgatc |
| 26.52 cM | 5605203 | A/C | S14151- | SEQ ID: 21 ccagcttcttttgctccatc | SEQ ID: 23 cattgtacgTccctc |
| | | | 1-Q1 | SEQ ID: 22 cgacgctcctaagtattggtg | SEQ ID: 24 atcattgtacgGccc |
| 26.52 cM | 5605275 | A/G | S14151- | SEQ ID: 26 aatcccacaccagcttctttt | SEQ ID: 28 cagaacaTcttggc |
| | | | 2-Q4 | SEQ ID: 27 gtgtggcactgtagcagataaagata | SEQ ID: 29 cagaacaCcttggc |
| 26.54 cM | 5608106 | A/G | S07164- | SEQ ID: 31 tcatttcctgatgctcaccata | SEQ ID: 33 ttgagaaaacGtctgca |
| | | | 1-Q12 | SEQ ID: 32 ggttgtatccatcttctgaactgc | SEQ ID: 34 ttgagaaaacAtctgca |
| 26.6 cM | 5630404 | A/G | S14182- | SEQ ID: 36 tgtactttggctgcgtctcc | SEQ ID: 38 ccatgtcaaTgcc |
| | | | 1-Q1 | SEQ ID: 37 ggtaactcctttgtaatgttcaccac | SEQ ID: 39 ccatgtcaaCgcca |
| 33.2 cM | 6754454 | C/G | 500812- | SEQ ID: 41 gctgctctttctctgctgtgatca | SEQ ID: 43 tataccCgtgagactat |
| | | | 1-A | SEQ ID: 42 tgggtggtttccttgtttataccaac | SEQ ID: 44 tataccGgtgagactat |
| 34.2 cM | 6671535 | A/G | S02780- | SEQ ID: 46 ggcatttgcttcaattttcc | SEQ ID: 48 actctggAtaacctg |
| | | | 1-A | SEQ ID: 47 acttttgcccctatakgatatgc | SEQ ID: 49 actctggGtaacctg |

Rag2 (LG-F)

| | | | | | |
|---|---|---|---|---|---|
| 72.08 | 28187733 | A/T | S01190- | SEQ ID: 127 ttcagctccccattatttcg | SEQ ID: 129 tcagctcaTttttgt |
| | | | 1-A | SEQ ID: 128 ttggccaacctatcctcaac | SEQ ID: 130 cagctcaCttttgt |
| 72.85 | 28829625 | A/G | 514761- | SEQ ID: 51 agagagcaacaaccagtaatttcata | SEQ ID: 53 ccactaaAgttagcctag |
| | | | 001- | SEQ ID: 52 acttagtgcatctattgcaaccac | SEQ ID: 54 ccactaaGgttagcctag |
| | | | Q001 | | |
| 72.85 | 28837383 | C/T | S14771- | SEQ ID: 56 ccttcaacaacagcagctttaat | SEQ ID: 58 cattagatcaacaCtgc |
| | | | 001- | SEQ ID: 57 ctgcttaatcgactgagctagacc | SEQ ID: 59 cattagatcaaacaTtgc |
| | | | Q001 | | |
| 73.0 cM | 29097652 | A/T | S07165- | SEQ ID: 61 gcttgtaagctattcccaaacg | SEQ ID: 63 tttcttatcTaaggttttg |
| | | | 1-Q3 | SEQ ID: 62 tatctgtgagcggttgcttg | SEQ ID: 64 ttcttatcAaaggttttg |
| 73.2 | 29678319 | C/T | S14778- | SEQ ID: 66 tgaggatatttatggaatttgtcaga | SEQ ID: 68 cttataaaacCgctttc |
| | | | 001- | SEQ ID: 67 catgatgagatcagaaaagaaatgc | SEQ ID: 69 cttataaaacTgcttttcc |
| | | | Q001 | | |
| 73.2 cM | 29825175 | C/G | S01164- | SEQ ID: 71 gacagtggagagttacgagga | SEQ ID: 73 ccacctacatCactac |
| | | | 1-Q1 | SEQ ID: 72 cacatctgaatcaccctgga | SEQ ID: 74 ccacctacatGactac |

Rag3 (JG-J)

| | | | | | |
|---|---|---|---|---|---|
| 37.8800 | 5140274 | A/G | S13662- | SEQ ID: 76 tctttatgatgatgagcagaagcta | SEQ ID: 78 ctttcagAgcattagc |
| | | | 1-Q3 | SEQ ID: 77 caccccaaaaacaaaacactc | SEQ ID: 79 tttgctttcagGgcat |
| | | | S13662- | SEQ ID: 80 gggaagagtctgaatggtgtct | SEQ ID: 82 ctttcagAgcattagc |
| | | | 1-Q6 | SEQ ID: 81 ccccaaaaacaaaacactcatc | SEQ ID: 83 tttgctttcagGgcat |
| 41.7323 | 5919650 | T/C | S13663- | SEQ ID: 85 tctgatgatgattatagtgggctct | SEQ ID: 87 ctgataacaaTagccc |
| | | | q-Q1 | SEQ ID: 86 tgctatgcatttgaaaccaca | SEQ ID: 88 ataacaaCagccctgact |
| 41.96 | 5960726 | C/G | S11411- | SEQ ID: 90 ggacccaacatcaatcaaatg | SEQ ID: 92 ttttctgCactccc |
| | | | 1-Q1 | SEQ ID: 91 tgcattctggaaagacatgg | SEQ ID: 93 ttttctgGactccc |
| 42.5533 | 6066531 | T/G | S13664- | SEQ ID: 95 catgccagtatgaatgtgctg | SEQ ID: 97 attgtgacactctatTgc |
| | | | 1-Q1 | SEQ ID: 96 tccgcacatttagttccctta | SEQ ID: 98 ttgtgacactctatGgca |
| | | | S13664- | SEQ ID: 99 caaagtgtcatgccagtatgaatg | SEQ ID: 101 attgtgacactctatTgc |
| | | | 1-Q002 | SEQ ID: 100 gttttattttcattccgcacatttag | SEQ ID: 102 ttgtgacactctatGgca |
| 42.9757 | 6231641 | G/A | S13672- | SEQ ID: 104 gatcggttcccaaactagca | SEQ ID: 106 cagttgattactCtgc |
| | | | 1-Q1 | SEQ ID: 105 aacatgcaaaatgcaccaag | SEQ ID: 107 cagttgattactTtgc |
| | | | S13672- | SEQ ID: 108 cggttcccaaactagcaggt | SEQ ID: 106 cagttgattactCtgc |
| | | | 1-Q2 | SEQ ID: 109 tgcaaaatgcaccaagttagat | SEQ ID: 107 cagttgattactTtgc |
| | | | S13672- | SEQ ID: 110 agatcggttcccaaactagcag | SEQ ID: 112 cagttgattactCtgc |
| | | | 1-Q3 | SEQ ID: 111 catgcaaaatgcaccaagtta | SEQ ID: 113 cagttgattactTtgc |
| 43.7295 | 6524877 | C/G | S13674- | SEQ ID: 115 ccaccattacccctctcctt | SEQ ID: 117 ttggcattcaGccc |
| | | | 1-Q1 | SEQ ID: 116 acctagcattgcaatctcttcc | SEQ ID: 118 tttggcattcaCccc |
| | | | S13674- | SEQ ID: 119 ttacccctctcctttctcaacatta | SEQ ID: 117 ttggcattcaGccc |
| | | | 1-Q007 | SEQ ID: 120 tgcaatctcttccaagctagaact | SEQ ID: 118 tttggcattcaCccc |
| 43.8186 | 6542422 | G/A | S13675- | SEQ ID: 122 aggtggtggcagtgttgatt | SEQ ID: 124 aaccgtggctCatt |
| | | | 2-Q1 | SEQ ID: 123 ctccaacatggctgtgctaa | SEQ ID: 125 caaaccgtggctTat |

Selected haplotypes that are based upon the markers in Table 1 are described in Table 2.

TABLE 2
Selected Rag haplotypes generated using the selected markers
Rag Haplotypes
Rag1

| S14161-1- | S09515-1- | S14151-2- | S07164-1- | |
|---|---|---|---|---|
| Q10 | Q1 | Q4 | Q12 | Haplotype |
| C | G | G | G | Rag1-a |
| G | A | G | A | Rag1-b |
| C, G | A | A | A | Rag1-c |
| C | G | A | A | Rag1-d |
| C | A | A | A | Rag1-e |
| G | A | A | A | Rag1-f |
| C | A, G | G | A | Rag1-g |

Rag2

| S07165-1- | S01190-1- | S01164-1- | |
|---|---|---|---|
| Q3 | A | Q1 | Haplotype |
| A | C | C | Rag2-a |
| T | T | G | Rag2-b |
| T | C | C | Rag2-c |
| A | T | G | Rag2-d |
| T | C | G | Rag2-f |

Rag3

| S11411-1- | S13674-1- | S13675-2- | |
|---|---|---|---|
| Q1 | Q1/Q007 | Q1 | Haplotype |
| C | C | G | Rag3-a |
| G | C | G | Rag3-b |
| G | G | A | Rag3-c |
| G | G | G | Rag3-d |

In addition to the markers discussed herein, information regarding useful soybean markers can be found, for example, on the USDA's Soybase website, available at www.soybase.org. One of skill in the art will recognize that the identification of favorable marker alleles may be germplasm-specific. The determination of which marker alleles correlate with resistance (or susceptibility) is determined for the particular germplasm under study. One of skill will also recognize that methods for identifying the favorable alleles are routine and well known in the art, and furthermore, that the identification and use of such favorable alleles is well within the scope of the invention.

In some examples marker profiles comprising two or more Rag haplotypes are provided. For instance, in one example, a particular Rag1 haplotype and a particular Rag2 haplotype define the marker profile of a particular plant. In another example, a particular Rag1 haplotype and a particular Rag3 haplotype define the marker profile of a particular plant. In a still further example, a particular Rag2 haplotype and a particular Rag3 haplotype define the marker profile of a particular plant. In an additional example, a particular Rag1 haplotype, a particular Rag2 haplotype, and a particular Rag3 haplotype define the marker profile of a particular plant. More specifically, a particular plant marker profile might be, for example, Rag1-a/Rag2-a or Rag1-b/Rag2-a/Rag3-c.

### Marker Assisted Selection:

The use of marker assisted selection (MAS) to select a soybean plant or germplasm which has a certain Rag haplotype or marker profile is provided. For instance, in certain examples a soybean plant or germplasm possessing a certain predetermined favorable Rag haplotype will be selected via MAS. In certain other examples, a soybean plant or germplasm possessing a certain predetermined favorable marker profile will be selected via MAS.

Using MAS, soybean plants or germplasm can be selected for markers or marker alleles that positively correlate with resistance, without actually raising soybean and measuring for resistance or improved resistance (or, contrawise, soybean plants can be selected against if they possess markers that negatively correlate with resistance or improved resistance). MAS is a powerful tool to select for desired phenotypes and for introgressing desired traits into cultivars of soybean (e.g., introgressing desired traits into elite lines). MAS is easily adapted to high throughput molecular analysis methods that can quickly screen large numbers of plant or germplasm genetic material for the markers of interest and is much more cost effective than raising and observing plants for visible traits.

### Nucleic Acid Amplification Methods:

In some examples, the molecular markers are detected using a suitable amplification-based detection method. In these types of methods, nucleic acid primers are typically hybridized to the conserved regions flanking the polymorphic marker region. In certain methods, nucleic acid probes that bind to the amplified region are also employed. In general, synthetic methods for making oligonucleotides, including primers and probes, are well known in the art. For example, oligonucleotides can be synthesized chemically according to the solid phase phosphoramidite triester method described by Beaucage and Caruthers (1981) Tetrahedron Letts 22:1859-1862, e.g., using a commercially available automated synthesizer, e.g., as described in Needham-VanDevanter, et al. (1984) Nucleic Acids Res. 12:6159-6168. Oligonucleotides, including modified oligonucleotides, can also be ordered from a variety of commercial sources known to persons of skill in the art.

It will be appreciated that suitable primers and probes to be used can be designed using any suitable method. It is not intended that the invention be limited to any particular primer, primer pair or probe. For example, primers can be designed using any suitable software program, such as LASERGENE® or Primer3.

It is not intended that the primers be limited to generating an amplicon of any particular size. For example, the primers used to amplify the marker loci and alleles herein are not limited to amplifying the entire region of the relevant locus. In some examples, marker amplification produces an amplicon at least 20 nucleotides in length, or alternatively, at least 50 nucleotides in length, or alternatively, at least 100 nucleotides in length, or alternatively, at least 200 nucleotides in length.

PCR, RT-PCR, and LCR are in particularly broad use as amplification and amplification-detection methods for amplifying nucleic acids of interest (e.g., those comprising marker loci), facilitating detection of the markers. Details regarding the use of these and other amplification methods are well known in the art and can be found in any of a variety of standard texts. Details for these techniques can also be found in numerous journal and patent references, such as Mullis, et al. (1987) U.S. Pat. No. 4,683,202; Arnheim & Levinson (Oct. 1, 1990) C&EN 36-47; Kwoh, et al. (1989) Proc. Natl. Acad. Sci. USA 86:1173; Guatelli, et al., (1990) Proc. Natl. Acad. Sci. USA87:1874; Lomeli, et al., (1989) J. Clin. Chem. 35:1826; Landegren, et al., (1988) Science 241:1077-1080; Van Brunt, (1990) Biotechnology 8:291-294; Wu and Wallace, (1989) Gene 4:560; Barringer, et al., (1990) Gene 89:117, and Sooknanan and Malek, (1995) Biotechnology 13:563-564.

Such nucleic acid amplification techniques can be applied to amplify and/or detect nucleic acids of interest, such as nucleic acids comprising marker loci. Amplification primers for amplifying useful marker loci and suitable probes to detect useful marker loci or to genotype SNP alleles are provided. For example, exemplary primers and probes are provided in Table 1, as are the target regions to which these primers and probes hybridize. However, one of skill will immediately recognize that other primer and probe sequences could also be used. For instance primers to either side of the given primers can be used in place of the given primers, so long as the primers can amplify a region that includes the allele to be detected, as can primers and probes directed to other SNP marker loci. Further, it will be appreciated that the precise probe to be used for detection can vary, e.g., any probe that can identify the region of a marker amplicon to be detected can be substituted for those examples provided herein. Further, the configuration of the amplification primers and detection probes can, of course, vary. Thus, the compositions and methods are not limited to the primers and probes specifically recited herein.

In certain examples, probes will possess a detectable label. Any suitable label can be used with a probe. Detectable labels suitable for use with nucleic acid probes include, for example, any composition detectable by spectroscopic, radioisotopic, photochemical, biochemical, immunochemical, electrical, optical, or chemical means. Useful labels include biotin for staining with labeled streptavidin conjugate, magnetic beads, fluorescent dyes, radiolabels, enzymes, and colorimetric labels. Other labels include ligands, which bind to antibodies labeled with fluorophores, chemiluminescent agents, and enzymes. A probe can also constitute radiolabelled PCR primers that are used to generate a radiolabelled amplicon. Labeling strategies for labeling nucleic acids and corresponding detection strategies can be found, e.g., in Haugland (1996) Handbook of Fluorescent Probes and Research Chemicals Sixth Edition by Molecular Probes, Inc. (Eugene Oreg.); or Haugland (2001) Handbook of Fluorescent Probes and Research Chemicals Eighth Edition by Molecular Probes, Inc. (Eugene Oreg.).

Detectable labels may also include reporter-quencher pairs, such as are employed in Molecular Beacon and TaqMan™ probes. The reporter may be a fluorescent organic dye modified with a suitable linking group for attachment to the oligonucleotide, such as to the terminal 3′ carbon or terminal 5′ carbon. The quencher may also be an organic dye, which may or may not be fluorescent, depending on the embodiment. Generally, whether the quencher is fluorescent or simply releases the transferred energy from the reporter by non-radiative decay, the absorption band of the quencher should at least substantially overlap the fluorescent emission band of the reporter to optimize the quenching. Non-fluorescent quenchers or dark quenchers typically function by absorbing energy from excited reporters, but do not release the energy radiatively.

Selection of appropriate reporter-quencher pairs for particular probes may be undertaken in accordance with known techniques. Fluorescent and dark quenchers and their relevant optical properties from which exemplary reporter-quencher pairs may be selected are listed and described, for example, in Berlman, Handbook of Fluorescence Spectra of Aromatic Molecules, 2nd ed., Academic Press, New York, 1971, the content of which is incorporated herein by reference. Examples of modifying reporters and quenchers for covalent attachment via common reactive groups that can be added to an oligonucleotide in the present invention may be found, for example, in Haugland, Handbook of Fluorescent Probes and Research Chemicals, Molecular Probes of Eugene, Oreg., 1992, the content of which is incorporated herein by reference.

In certain examples, reporter-quencher pairs are selected from xanthene dyes including fluoresceins and rhodamine dyes. Many suitable forms of these compounds are available commercially with substituents on the phenyl groups, which can be used as the site for bonding or as the bonding functionality for attachment to an oligonucleotide. Another useful group of fluorescent compounds for use as reporters are the naphthylamines, having an amino group in the alpha or beta position. Included among such naphthylamino compounds are 1-dimethylaminonaphthyl-5 sulfonate, 1-anilino-8-naphthalene sulfonate and 2-p-touidinyl-6-naphthalene sulfonate. Other dyes include 3-phenyl-7-isocyanatocoumarin; acridines such as 9-isothiocyanatoacridine; N-(p-(2-benzoxazolyl)phenyl)maleimide; benzoxadiazoles; stilbenes; pyrenes and the like. In certain other examples, the reporters and quenchers are selected from fluorescein and rhodamine dyes. These dyes and appropriate linking methodologies for attachment to oligonucleotides are well known in the art.

Suitable examples of reporters may be selected from dyes such as SYBR green, 5-carboxyfluorescein (5-FAM™ available from Applied Biosystems of Foster City, Calif.), 6-carboxyfluorescein (6-FAM), tetrachloro-6-carboxyfluorescein (TET), 2,7-dimethoxy-4,5-dichloro-6-carboxyfluorescein, hexachloro-6-carboxyfluorescein (HEX), 6-carboxy-2′,4,7,7′-tetrachlorofluorescein (6-TET™ available from Applied Biosystems), carboxy-X-rhodamine (ROX), 6-carboxy-4′,5′-dichloro-2′,7′-dimethoxyfluorescein (6-JOE™ available from Applied Biosystems), VIC™ dye products available from Molecular Probes, Inc., NED™ dye products available from available from Applied Biosystems, and the like. Suitable examples of quenchers may be selected from 6-carboxy-tetramethyl-rhodamine, 4-(4-dimethylaminophenylazo) benzoic acid (DABYL), tetramethylrhodamine (TAMRA), BHQ-0™, BHQ-1™, BHQ-2™, and BHQ-3™, each of which are available from Biosearch Technologies, Inc. of Novato, Calif., QSY-7™, QSY-9™, QSY-21™ and QSY35™, each of which are available from Molecular Probes, Inc., and the like.

In one aspect, real time PCR or LCR is performed on the amplification mixtures described herein, e.g., using molecular beacons or TaqMan™ probes. A molecular beacon (MB) is an oligonucleotide which, under appropriate hybridization conditions, self-hybridizes to form a stem and loop structure. The MB has a label and a quencher at the termini of the oligonucleotide; thus, under conditions that permit intra-molecular hybridization, the label is typically quenched (or at least altered in its fluorescence) by the quencher. Under conditions where the MB does not display intra-molecular hybridization (e.g., when bound to a target nucleic acid, such as to a region of an amplicon during amplification), the MB label is unquenched. Details regarding standard methods of making and using MBs are well established in the literature and MBs are available from a number of commercial reagent sources. See also, e.g., Leone, et al., (1995) Molecular beacon probes combined with amplification by NASBA enable homogenous real-time detection of RNA, Nucleic Acids Res. 26:2150-2155; Tyagi and Kramer, (1996) Molecular beacons: probes that fluoresce upon hybridization, Nature Biotechnology 14:303-308; Blok and Kramer, (1997) Amplifiable hybridization probes containing a molecular switch, Mol Cell Probes 11:187-194; Hsuih. et al., (1997) Novel, ligation-dependent PCR assay for detection of hepatitis C in serum, J Clin Microbiol 34:501-507; Kostrikis, et al., (1998) Molecular beacons: spectral genotyping of human alleles, Science 279:1228-1229; Sokol, et al., (1998) Real time detection of DNA:RNA hybridization in living cells, Proc. Natl. Acad. Sci. U.S.A. 95:11538-11543; Tyagi, et al., (1998) Multicolor molecular beacons for allele discrimination, Nature Biotechnology 16:49-53; Bonnet, et al., (1999) Thermodynamic basis of the chemical specificity of structured DNA probes, Proc. Natl. Acad. Sci. U.S.A. 96:6171-6176; Fang, et al. (1999) Designing a novel molecular beacon for surface-immobilized DNA hybridization studies, J. Am. Chem. Soc. 121:2921-2922; Marras, et al., (1999) Multiplex detection of single-nucleotide variation using molecular beacons, Genet. Anal. Biomol. Eng. 14:151-156; and Vet, et al., (1999) Multiplex detection of four pathogenic retroviruses using molecular beacons, Proc. Natl. Acad. Sci. U.S.A. 96:6394-6399. Additional details regarding MB construction and use is found in the patent literature, e.g., U.S. Pat. Nos. 5,925,517; 6,150,097; and 6,037,130.

Another real-time detection method is the 5′-exonuclease detection method, also called the TaqMan™ assay, as set forth in U.S. Pat. Nos. 5,804,375; 5,538,848; 5,487,972; and 5,210,015, each of which is hereby incorporated by reference in its entirety. In the TaqMan™ assay, a modified probe, typically 10-25 nucleic acids in length, is employed during PCR which binds intermediate to or between the two members of the amplification primer pair. The modified probe possesses a reporter and a quencher and is designed to generate a detectable signal to indicate that it has hybridized with the target nucleic acid sequence during PCR. As long as both the reporter and the quencher are on the probe, the quencher stops the reporter from emitting a detectable signal. However, as the polymerase extends the primer during amplification, the intrinsic 5′ to 3′ nuclease activity of the polymerase degrades the probe, separating the reporter from the quencher, and enabling the detectable signal to be emitted. Generally, the amount of detectable signal generated during the amplification cycle is proportional to the amount of product generated in each cycle.

It is well known that the efficiency of quenching is a strong function of the proximity of the reporter and the quencher, i.e., as the two molecules get closer, the quenching efficiency increases. As quenching is strongly dependent on the physical proximity of the reporter and quencher, the reporter and the quencher are preferably attached to the probe within a few nucleotides of one another, usually within 30 nucleotides of one another, more preferably with a separation of from about 6 to 16 nucleotides. Typically, this separation is achieved by attaching one member of a reporter-quencher pair to the 5′ end of the probe and the other member to a nucleotide about 6 to 16 nucleotides away, in some cases at the 3′ end of the probe.

Separate detection probes can also be omitted in amplification/detection methods, e.g., by performing a real time amplification reaction that detects product formation by modification of the relevant amplification primer upon incorporation into a product, incorporation of labeled nucleotides into an amplicon, or by monitoring changes in molecular rotation properties of amplicons as compared to unamplified precursors (e.g., by fluorescence polarization).

Further, it will be appreciated that amplification is not a requirement for marker detection—for example, one can directly detect unamplified genomic DNA simply by performing a Southern blot on a sample of genomic DNA. Procedures for performing Southern blotting, amplification e.g., (PCR, LCR, or the like), and many other nucleic acid detection methods are well established and are taught, e.g., in Sambrook, et al., Molecular Cloning—A Laboratory Manual (3d ed.), Vol. 1-3, Cold Spring Harbor Laboratory, Cold Spring Harbor, N.Y., 2000 (“Sambrook”); Current Protocols in Molecular Biology, F. M. Ausubel, et al., eds., Current Protocols, a joint venture between Greene Publishing Associates, Inc. and John Wiley & Sons, Inc., (supplemented through 2002) (“Ausubel”)) and PCR Protocols A Guide to Methods and Applications (Innis, et al., eds) Academic Press Inc. San Diego, Calif. (1990) (Innis). Additional details regarding detection of nucleic acids in plants can also be found, e.g., in Plant Molecular Biology (1993) Croy (ed.) BIOS Scientific Publishers, Inc.

Other techniques for detecting SNPs can also be employed, such as allele specific hybridization (ASH). ASH technology is based on the stable annealing of a short, single-stranded, oligonucleotide probe to a completely complementary single-stranded target nucleic acid. Detection is via an isotopic or non-isotopic label attached to the probe. For each polymorphism, two or more different ASH probes are designed to have identical DNA sequences except at the polymorphic nucleotides. Each probe will have exact homology with one allele sequence so that the range of probes can distinguish all the known alternative allele sequences. Each probe is hybridized to the target DNA. With appropriate probe design and hybridization conditions, a single-base mismatch between the probe and target DNA will prevent hybridization.

### Real-Time SNP Detection Assays:

Real-time amplification assays, including MB or TaqMan™ based assays, are especially useful for detecting SNP alleles. In such cases, probes are typically designed to bind to the amplicon region that includes the SNP locus, with one allele-specific probe being designed for each possible SNP allele. For instance, if there are two known SNP alleles for a particular SNP locus, “A” or “C,” then one probe is designed with an “A” at the SNP position, while a separate probe is designed with a “C” at the SNP position. While the probes are typically identical to one another other than at the SNP position, they need not be. For instance, the two allele-specific probes could be shifted upstream or downstream relative to one another by one or more bases. However, if the probes are not otherwise identical, they should be designed such that they bind with approximately equal efficiencies, which can be accomplished by designing under a strict set of parameters that restrict the chemical properties of the probes. Further, a different detectable label, for instance a different reporter-quencher pair, is typically employed on each different allele-specific probe to permit differential detection of each probe. In certain examples, each allele-specific probe for a certain SNP locus is 11-20 nucleotides in length, dual-labeled with a florescence quencher at the 3′ end and either the 6-FAM (6-carboxyfluorescein) or VIC (4,7,2′-trichloro-7′-phenyl-6-carboxyfluorescein) fluorophore at the 5′ end.

To effectuate SNP allele detection, a real-time PCR reaction can be performed using primers that amplify the region including the SNP locus, for instance the target regions listed in Table 1, the reaction being performed in the presence of all allele-specific probes for the given SNP locus. By then detecting signal for each detectable label employed and determining which detectable label(s) demonstrated an increased signal, a determination can be made of which allele-specific probe(s) bound to the amplicon and, thus, which SNP allele(s) the amplicon possessed. For instance, when 6-FAM- and VIC-labeled probes are employed, the distinct emission wavelengths of 6-FAM (518 nm) and VIC (554 nm) can be captured. A sample that is homozygous for one allele will have fluorescence from only the respective 6-FAM or VIC fluorophore, while a sample that is heterozygous at the analyzed locus will have both 6-FAM and VIC fluorescence.

The KASPar® and Illumina® Detection Systems are additional examples of commercially-available marker detection systems. KASPar® is a homogeneous fluorescent genotyping system which utilizes allele specific hybridization and a unique form of allele specific PCR (primer extension) in order to identify genetic markers (e.g. a particular SNP locus associated with aphid resistance). Illumina® detection systems utilize similar technology in a fixed platform format. The fixed platform utilizes a physical plate that can be created with up to 384 markers. The Illumina® system is created with a single set of markers that cannot be changed and utilizes dyes to indicate marker detection.

These systems and methods represent a wide variety of available detection methods which can be utilized to detect markers associated with improved aphid resistance, but any other suitable method could also be used.

### Introgression:

Introgression of soybean aphid resistance into non-resistant or less-resistant soybean germplasm is provided. Any method for introgressing a QTL or marker into soybean plants known to one of skill in the art can be used. Typically, a first soybean germplasm that contains resistance to soybean aphid derived from a particular Rag haplotype or marker profile and a second soybean germplasm that lacks such resistance derived from the Rag haplotype or marker profile are provided. The first soybean germplasm may be crossed with the second soybean germplasm to provide progeny soybean germplasm. These progeny germplasm are screened to determine the presence of soybean aphid resistance derived from the Rag haplotype or marker profile, and progeny that tests positive for the presence of resistance derived from the Rag haplotype or marker profile are selected as being soybean germplasm into which the Rag haplotype or marker profile has been introgressed. Methods for performing such screening are well known in the art and any suitable method can be used.

Introgression of Favorable Alleles—Efficient Backcrossing of Resistance Markers into Elite Lines:

One application of MAS is to use the resistance or improved resistance markers, haplotypes or marker profiles to increase the efficiency of an introgression or backcrossing effort aimed at introducing a resistance trait into a desired (typically high yielding) background. In marker assisted backcrossing of specific markers from a donor source, e.g., to an elite genetic background, one selects among backcross progeny for the donor trait and then uses repeated backcrossing to the elite line to reconstitute as much of the elite background's genome as possible.

Thus, the markers and methods can be utilized to guide marker assisted selection or breeding of soybean varieties with the desired complement (set) of allelic forms of chromosome segments associated with superior agronomic performance (resistance, along with any other available markers for yield, disease resistance, etc.). Any of the disclosed marker alleles, haplotypes, or marker profiles can be introduced into a soybean line via introgression, by traditional breeding (or introduced via transformation, or both) to yield a soybean plant with superior agronomic performance. The number of alleles associated with resistance that can be introduced or be present in a soybean plant ranges from 1 to the number of alleles disclosed herein, each integer of which is incorporated herein as if explicitly recited.

This also provides a method of making a progeny soybean plant and these progeny soybean plants, per se. The method comprises crossing a first parent soybean plant with a second soybean plant and growing the female soybean plant under plant growth conditions to yield soybean plant progeny. Methods of crossing and growing soybean plants are well within the ability of those of ordinary skill in the art. Such soybean plant progeny can be assayed for alleles associated with resistance and, thereby, the desired progeny selected. Such progeny plants or seed can be sold commercially for soybean production, used for food, processed to obtain a desired constituent of the soybean, or further utilized in subsequent rounds of breeding. At least one of the first or second soybean plants is a soybean plant in that it comprises at least one of the Rag haplotypes or marker profiles, such that the progeny are capable of inheriting the haplotype o marker profile.

Often, a method is applied to at least one related soybean plant such as from progenitor or descendant lines in the subject soybean plants pedigree such that inheritance of the desired resistance can be traced. The number of generations separating the soybean plants being subject to the methods of the present invention will generally be from 1 to 20, commonly 1 to 5, and typically 1, 2, or 3 generations of separation, and quite often a direct descendant or parent of the soybean plant will be subject to the method (i.e., 1 generation of separation).

Introgression of Favorable Alleles—Incorporation of “Exotic” Germplasm while Maintaining Breeding Progress:

Genetic diversity is important for long term genetic gain in any breeding program. With limited diversity, genetic gain will eventually plateau when all of the favorable alleles have been fixed within the elite population. One objective is to incorporate diversity into an elite pool without losing the genetic gain that has already been made and with the minimum possible investment. MAS provides an indication of which genomic regions and which favorable alleles from the original ancestors have been selected for and conserved over time, facilitating efforts to incorporate favorable variation from exotic germplasm sources (parents that are unrelated to the elite gene pool) in the hopes of finding favorable alleles that do not currently exist in the elite gene pool.

For example, the markers, haplotypes, primers, probes, and marker profiles can be used for MAS in crosses involving elite x exotic soybean lines by subjecting the segregating progeny to MAS to maintain major yield alleles, along with the resistance marker alleles herein.

### Transgenic Approaches:

As an alternative to standard breeding methods of introducing traits of interest into soybean (e.g., introgression), transgenic approaches can also be used to create transgenic plants with the desired traits. In these methods, exogenous nucleic acids that encode a desired Rag haplotype or marker profile are introduced into target plants or germplasm. For example, a nucleic acid that codes for a resistance trait is cloned, e.g., via positional cloning, and introduced into a target plant or germplasm.

### Phenotypic Screening for Soybean Aphid Resistant Soybean Plants:

Three types of soybean aphid resistance have been described: antibiosis, antixenosis, and tolerance. Experienced plant breeders can recognize resistant soybean plants in the field, and can select the resistant individuals or populations for breeding purposes or for propagation. In this context, the plant breeder recognizes “resistant” and “non-resistant” or “susceptible” soybean plants. However, plant resistance is a phenotypic spectrum consisting of extremes in resistance and susceptibility, as well as a continuum of intermediate resistance phenotypes. Evaluation of these intermediate phenotypes using reproducible assays are of value to scientists who seek to identify genetic loci that impart resistance, to conduct marker assisted selection for resistance populations, and to use introgression techniques to breed a resistance trait into an elite soybean line, for example.

To that end, screening and selection of resistant soybean plants may be performed, for example, by exposing plants to soybean aphid in a live aphid assay and selecting those plants showing resistance to aphids. The live aphid assay may be any such assay known to the art, e.g., as described in Hill, C. B., et al., Resistance to the soybean aphid in soybean germplasm, (2004) Crop Science 44:98-106, Hill, C. B., et al., Resistance of Glycine species and various cultivated legumes to the soybean aphid (Homoptera: Aphididae), (2004) J. Economic Entomology 97:1071-1077, or Li, Y., et al., Effect of three resistant soybean genotypes on the tecunalry, mortality, and maturation of soybean aphid (Homoptera: Aphididae), (2004) J. Economic Entomology 97:1106-1111, or as described in the Examples hereof.

One example of an antixenosis resistance assay includes placing aphids or aphid-infested plant parts on VC or V1 stage plants and rating aphid population and plant damage weekly. For example, in certain examples, numerous viviparous alate adult females are placed on newly expanded unifoliates with a moistened camel's hair paintbrush, the plants are arranged in a randomized design within a tray, and the aphid resistance is evaluated at 7 and 14 days after infestation, using an antixenosis rating scale. One example of such an antixenosis scale is a 1-9 rating scale wherein:
9=Equivalent or better when compared to a resistant check—No aphids on the plant;
7=Very little damage, only a few aphids found on the plant;
5=Moderately Susceptible;
3=Major damage, including stunting and foliar stress; and
1=Plants are completely covered—Severe damage, including severe stunting and necrosis; equivalent or worse when compared to a susceptible check.

One example of an antibiosis resistance assay includes placing one double-sided sticky cage containing two alate adult females on each unifoliate of plants at the V1 stage and then placing a piece of organdy cloth over the cage to restrict the aphids' movements. This is done for both the plant variety to be tested and a plant variety known to be susceptible. The aphids are then allowed to reproduce for 96 hours and, at the end of this period, the cages are removed and counts performed on the surviving and deceased aphids to determine the antibiosis resistance of the plants tested. Plants with a high rate of nymphal production are classified as susceptible. Plants with some nymphs, but with statistically lower nymphal populations compared to the susceptible check are classified as moderately resistant. Plants with no nymph production within the sticky cages and dead or unhealthy in appearance adults are classified as resistant.

### Automated Detection/Correlation Systems, Kits, and Nucleic Acids:

In some examples, a kit or an automated system for detecting markers, Rag haplotypes, and marker profiles, and/or correlating the markers, Rag haplotypes, and marker profiles with a desired phenotype (e.g., resistance) are provided. Thus, a typical kit or system can include a set of marker probes or primers configured to detect at least one favorable allele of one or more marker locus associated with resistance or improved resistance to a soybean aphid infestation, for instance a favorable Rag haplotype or marker profile. These probes or primers can be configured, for example, to detect the marker alleles noted in the tables and examples herein, e.g., using any available allele detection format, such as solid or liquid phase array based detection, microfluidic-based sample detection, etc. The systems and kits can further include packaging materials for packaging the probes, primers, or instructions, controls such as control amplification reactions that include probes, primers or template nucleic acids for amplifications, molecular size markers, or the like.

A typical system can also include a detector that is configured to detect one or more signal outputs from the set of marker probes or primers, or amplicon thereof, thereby identifying the presence or absence of the allele. A wide variety of signal detection apparatus are available, including photo multiplier tubes, spectrophotometers, CCD arrays, scanning detectors, phototubes and photodiodes, microscope stations, galvo-scans, microfluidic nucleic acid amplification detection appliances and the like. The precise configuration of the detector will depend, in part, on the type of label used to detect the marker allele, as well as the instrumentation that is most conveniently obtained for the user. Detectors that detect fluorescence, phosphorescence, radioactivity, pH, charge, absorbance, luminescence, temperature, magnetism or the like can be used. Typical detector examples include light (e.g., fluorescence) detectors or radioactivity detectors. For example, detection of a light emission (e.g., a fluorescence emission) or other probe label is indicative of the presence or absence of a marker allele. Fluorescent detection is generally used for detection of amplified nucleic acids (however, upstream and/or downstream operations can also be performed on amplicons, which can involve other detection methods). In general, the detector detects one or more label (e.g., light) emission from a probe label, which is indicative of the presence or absence of a marker allele. The detector(s) optionally monitors one or a plurality of signals from an amplification reaction. For example, the detector can monitor optical signals which correspond to “real time” amplification assay results.

System or kit instructions that describe how to use the system or kit or that correlate the presence or absence of the favorable allele with the predicted resistance are also provided. For example, the instructions can include at least one look-up table that includes a correlation between the presence or absence of the favorable alleles, haplotypes, or marker profiles and the predicted resistance or improved resistance. The precise form of the instructions can vary depending on the components of the system, e.g., they can be present as system software in one or more integrated unit of the system (e.g., a microprocessor, computer or computer readable medium), or can be present in one or more units (e.g., computers or computer readable media) operably coupled to the detector. As noted, in one typical example, the system instructions include at least one look-up table that includes a correlation between the presence or absence of the favorable alleles and predicted resistance or improved resistance. The instructions also typically include instructions providing a user interface with the system, e.g., to permit a user to view results of a sample analysis and to input parameters into the system.

Isolated nucleic acids comprising a nucleic acid sequence coding for resistance to soybean aphid, or sequences complementary thereto, are also included. In certain examples, the isolated nucleic acids are capable of hybridizing under stringent conditions to nucleic acids of a soybean cultivar resistant to soybean, for instance to particular SNPs that comprise a Rag haplotype or marker profile. Vectors comprising such nucleic acids, expression products of such vectors expressed in a host compatible therewith, antibodies to the expression product (both polyclonal and monoclonal), and antisense nucleic acids are also included.

As the parental line having soybean aphid resistance, any line known to the art or disclosed herein may be used. Also included are soybean plants produced by any of the foregoing methods. Seed of a soybean germplasm produced by crossing a soybean variety having a Rag haplotype or marker profile associated with soybean aphid resistance with a soybean variety lacking such Rag haplotype or marker profile, and progeny thereof, is also included.

The present invention is illustrated by the following examples. The foregoing and following description of the present invention and the various examples are not intended to be limiting of the invention but rather are illustrative thereof. Hence, it will be understood that the invention is not limited to the specific details of these examples.

### EXAMPLES

### Example 1

### Phenotyping

### Aphid Isolates:

The three biotype colonies for soybean aphid are maintained in a growth chamber at the Dallas Center Containment Facility (Dallas Center, Iowa). The colonies are maintained on a continuous supply of soybean variety 90M60. Two colonies of Urbana, Ill. (biotype 1) and Wooster, Ohio (biotype 2) were obtained from Brian Diers at the University of Illinois. Lime Springs, Iowa (biotype X) was collected from soybean fields in Limes Springs, Iowa. The colonies are maintained in isolated tents to avoid mixing.

### Experiments:

Thirty five hundred soybean plant introductions (PIs) from Maturity Groups I to X were obtained from the USDA Soybean Germplasm Collection in Urbana, Ill. All 3500 PIs were evaluated in the aphid antixenosis screen using biotype 2, which was selected because it had overcome the Rag1 resistance. As such, PIs containing only Rag1 resistance can be avoided by using this isolate. The soybean cultivar 93B15 was used as a susceptible check in all bioassay experiments and a Rag1 and a Rag2 donor line were used as the resistant checks.

### Choice Bioassay (Antixenosis):

The choice tests were conducted in a growth chamber with temperatures between 22 and 25° C. with a 16 hour photoperiod. All PIs were first screened in the antixenosis bioassay to identify PIs that the aphids did not prefer. Five reps of each variety were planted in Cone-tainers™ (Stuewe and Sons, Inc., Tangent, Oreg.) and infested at the V1 growth stage. Seven viviparous alate adult females were placed on the newly expanded unifoliates with a moistened camel's hair paintbrush. The plants were arranged in completely randomized design within a tray including the five replicates and the susceptible and resistant checks. The trays were watered from the bottom to avoid disrupting the aphid feeding. The aphid resistance was evaluated at 7 and 14 days after infestation, using a 1-9 antixenosis rating. The resistant plants were then screened in the antibiosis bioassay.

### Antixenosis Scale:

9=Equivalent or better when compared to the resistant check—No aphids on the plant
7=Very little damage, only a few aphids found on the plant.

### 5=Moderately Susceptible

3=Major damage, including stunting and foliar stress
1=Plants are completely covered. Severe damage, including severe stunting and necrosis; equivalent or worse when compared to the susceptible

### Non-Choice Bioassay (Antibiosis):

To determine antibiosis resistance, a non-choice test was conducted (i.e., a test wherein the aphids have no choice but to either feed on the plant or starve to death). The non-choice bioassay was conducted using the same environmental conditions as described above in the choice bioassay. At the V1 stage, one double-sided sticky cage was placed on each unifoliate. Using a moistened paintbrush, two viviparous alate adult females were placed within the cage and a piece of organdy cloth was placed over the cage to restrict the aphids' movements. Five replicates of each variety and a susceptible variety check (93B15) were arranged in completely randomized design within a tray. The aphids were allowed to reproduce for 96 hours and then the survival, death, and fecundity of the aphids within the cage were recorded at 96 hours. The fecundity was calculated as the mean number of surviving nymphs produced within a cage during the 96 hour period for each plant introduction. Plants that had a high rate of nymphal production were classified as susceptible. Plants with some nymphs, but with statistically lower populations compared to the susceptible check were classified as moderately resistant. Plants with no nymph production within the sticky cages and dead or unhealthy in appearance adults were classified as resistant.

### Confirmation of Aphid Biotype Resistance:

The plants identified as resistant in the choice and non-choice bioassay using biotype 2 were then screened using the additional biotypes (biotype 1, 3, and X) to determine the biotype profile. The plants that exhibited the strongest aphid resistance were crossed in a growth chamber. Numerous plant introductions were identified with either antixenosis or antibiosis aphid resistance to all three biotypes, as shown in Table 3 (S=susceptible; R=resistant; M=moderately resistant).

TABLE 3
Aphid resistance phenotype and genotype data for selected PIs

| | Maturity | Country | Antibiosis Results | | | | Antixenosis Results | | | |
|---|---|---|---|---|---|---|---|---|---|---|
| Variety | Group | of Origin | Bio. 1 | Bio. 2 | Bio. 3 | Bio. X | Bio. 1 | Bio. 2 | Bio. 3 | Bio. X |
| PI567666 | IV | China | R | R | R | R | R | R | R | R |
| PI567622 | IV | China | R | R | R | R | R | R | R | R |
| PI219652 | VII | Indonesia | S | S | S | S | R | R | R | R |
| PI219655 | VII | Indonesia | S | S | S | S | R | R | R | R |
| 95B97 | | Pioneer | R | S | M | R | R | S | M | R |
| Dowling | VIII | US | R | S | M | R | R | S | M | R |
| LD08-89068a | III | Illinois | R | R | S | S | R | R | S | S |
| PI200538 | VIII | Japan | R | R | S | S | R | R | S | S |
| JACKSON | VII | US | R | S | R | R | R | S | R | R |
| PI567541B | III | China | S | S | S | S | R | R | S | S |
| PI567597C | III | China | R | R | R | S | R | R | R | R |
| P1567543C | III | China | S | S | S | S | R | S | R | R |
| PI243540 | IV | Japan | R | R | S | S | R | R | S | S |
| PI587577E | V | China | R | R | R | R | R | R | R | R |
| PI587973B | V | China | R | R | R | R | R | R | R | R |
| PI567392 | V | China | R | R | S | S | R | R | S | S |
| PI567055 | VIII | Indonesia | R | R | R | R | R | R | R | R |
| PI567063 | VII | Indonesia | S | S | S | S | R | R | R | R |
| FC031416 | VII | Unknown | M | M | R | R | R | M | R | R |
| PI507089B | IV | Japan | R | R | M | S | R | R | M | S |
| PI567183 | V | Vietnam | R | R | R | R | R | M | R | R |

### Phenotyping the Mapping Population for Aphid Resistance:

The resistant PIs were crossed with seven elite parents to generate mapping populations. The F1 plants were phenotyped for aphid resistance. The segregating F2 plants from the same cross were phenotyped using the Ohio isolate (biotype 2) in the choice and non-choice bioassay. The individual plants were grown in Cone-tainers™. Five Cone-tainers™ of each of the two parents were placed within the racks filled with the infested F2 plants. One week after infestation, the plants were evaluated and rated for aphid resistance. 180 plants were leaf punched and collected in 2-ml tubes and placed within collection plates. The tissue was then lyophilized.

### Example 2

### Genotyping

For genotypic data, DNA was isolated from the collected leaves. Leaf tissue was punched and the tissue was genotyped using SNP markers within or linked to each of the three Rag loci: Rag1, Rag2, and Rag3. The specific markers that were examined and relevant information for each of those SNP markers is presented in Table 1. Based on the results of this SNP marker analysis, and using the Rag haplotype information presented in Table 2, each of the selected PIs was assigned a Rag haplotype for each of Rag1, Rag2, and Rag3, if applicable, which, when taken together, defined a marker profile for each PI. The results of this analysis are presented in Table 4.

TABLE 4
Rag haplotype and marker profile genotype data for selected PIs

| | Rag Haplotype/ |
|---|---|
| Variety | Marker Profile |
| PI567666 | Rag1-b & Rag3-b |
| PI567622 | Rag1-b & Rag3-b |
| PI219652 | Rag1-c & Rag3-d |
| PI219655 | Rag1-c & Rag3-d |
| 95B97 | Rag1-a |
| Dowling | Rag1-a |
| LD08-89068a | Rag2-b |
| PI200538 | Rag2-b |
| JACKSON | Rag1-a |
| PI567541B | Rag1-b & Rag2-c |
| PI567597C | Rag3-b |
| PI567543C | Rag3-a |
| PI243540 | Rag2-a |
| PI587577E | Rag1-b |
| PI587973B | Rag1-b |
| PI567392 | Rag2-c |
| PI567055 | Rag1-c & Rag3-d |
| PI567063 | Rag1-c & Rag3-d |
| FC031416 | Rag1-e |
| PI507089B | Rag2-d |
| PI567183 | Rag1-d & Rag2-c |

These results demonstrate that a change in one Rag haplotype, which can in some cases be a simple change in one SNP marker, can result in phenotypic changes in aphid resistance. For instance, PI567541B, which is Rag1-b and Rag2-c, only has antixenosis resistance to Biotypes 1 and 2 and no antibiosis resistance to any biotypes, while PI567183, which is both Rag1-d and Rag2-c, shows at least moderate antibiosis and antixenosis resistant to all four biotypes.

What is claimed is:

1. A method of identifying a first soybean plant or germplasm that displays improved resistance to one or more soybean aphid biotypes, the improved resistance comprising one or more of improved antibiosis resistance and improved antixenosis resistance, the method comprising detecting in the first soybean plant or germplasm, or a part thereof, at least one Rag haplotype that is associated with the improved soybean aphid resistance, the at least one Rag haplotype comprising marker loci selected from the group consisting of:
(a) one or more marker loci selected from the group consisting S14181-1-Q1, S13871-1-Q1, S14161-1-Q10, S09515-1-Q1, S14151-1-Q1, S14151-2-Q4, S07164-1-Q12, S14182-1-Q1, S00812-1-A, and S02780-1-A;
(b) one or more marker loci selected from the group consisting of S01190-1-A, S14761-001-Q001, S14771-001-Q001, S07165-1-Q3, S14778-001-Q001, and S01164-1-Q1;
(c) one or more marker loci selected from the group consisting of S13662-1-Q3/Q6, S13663-1-Q1, S11411-1-Q1, S13664-1-Q1/Q002, S13672-1-Q1/Q2/Q3, S13674-1-Q1/Q007, and S13675-2-Q1.
(d) one or more SNP loci located at physical positions 5516385, 5516818, 5598980, 5602544, 5605203, 5605275, 5608106, 5630404, 6754454, and 6671535 on LG-M of the soybean genome;
(e) one or more SNP loci located at physical positions 28187733, 28829625, 28837383, 29097652, 29678319, and 29825175 on LG-F of the soybean genome; and
(f) one or more SNP loci located at physical positions 5140274, 5919650, 5960726, 6066531, 6231641, 6524877, and 6542422 on LG-J of the soybean genome.

2. The method of claim 1, wherein the improved soybean aphid resistance comprises both improved antibiosis resistance and improved antixenosis resistance.

3. The method of claim 1, wherein the improved soybean aphid resistance comprises improved resistance to at least two of soybean aphid biotypes 1, 2, 3, and X.

4. The method of claim 1, wherein the at least one Rag haplotype detected comprises two or more of the marker loci within one or more of (a), (b), or (c).

5. The method of claim 1, wherein the at least one Rag haplotype comprises one or more of:
(a) the marker loci S14161-1-Q10, S09515-1-Q1, S14151-2-Q4, and S07164-1-Q12;
(b) the marker loci S07165-1-Q3, S01190-1-A, and S01164-1-Q1; or
(c) the marker loci S11411-1-Q1, S13674-1-Q1/Q007, and S13675-2-Q1.

6. The method of claim 1, further comprising detecting a marker profile comprising two or more of the Rag haplotypes of (a), (b), or (c).

7. The method of claim 5, further comprising detecting a marker profile comprising two or more of the Rag haplotypes of (a), (b), or (c).

8. The method of claim 1, wherein the germplasm is a soybean variety.

9. The method of claim 1, wherein the detecting comprises amplifying at least one of said marker loci or a portion thereof and detecting the resulting amplified marker amplicon.

10. The method of claim 9, wherein the amplifying comprises:
a) admixing an amplification primer or amplification primer pair for each marker locus being amplified with a nucleic acid isolated from the first soybean plant or germplasm, wherein the primer or primer pair is complementary or partially complementary to at least a portion of the marker locus, and is capable of initiating DNA polymerization by a DNA polymerase using the soybean nucleic acid as a template; and
b) extending the primer or primer pair in a DNA polymerization reaction comprising a DNA polymerase and a template nucleic acid to generate at least one amplicon.

11. The method of claim 10, wherein said method comprises amplifying at least a portion of one or more genome regions selected from the group consisting of SEQ ID NOs: 5, 10, 15, 20, 25, 30, 35, 40, 45, 50, 55, 60, 65, 70, 75, 84, 89, 94, 103, 114, 121, 126, and 131.

12. The method of claim 10, wherein said primer or primer pair comprises a nucleic acid sequence selected from the group consisting of SEQ ID NOs: 1, 2, 6, 7, 11, 12, 16, 17, 21, 22, 26, 27, 31, 32, 36, 37, 41, 42, 46, 47, 51, 52, 56, 57, 61, 62, 66, 67, 71, 72, 76, 77, 80, 81, 85, 86, 90, 91, 95, 96, 99, 100, 104, 105, 108, 109, 110, 111, 115, 116, 119, 120, 122, 123, 127, and 128.

13. The method of claim 10, wherein the method further comprises providing one or more labeled nucleic acid probes suitable for detection of each marker locus being amplified.

14. The method of claim 13, wherein said labeled nucleic acid probe comprises a nucleic acid sequence selected from the group consisting of SEQ ID NOs: 3, 4, 8, 9, 13, 14, 18, 19, 23, 24, 28, 29, 33, 34, 38, 39, 43, 44, 48, 49, 53, 54, 58, 59, 63, 64, 68, 69, 73, 74, 78, 79, 82, 83, 87, 88, 92, 93, 97, 98, 101, 102, 106, 107, 112, 113, 117, 118, 124, 125, 129, 130, 5, 10, 15, 20, 25, 30, 35, 40, 45, 50, 55, 60, 65, 70, 75, 84, 89, 94, 103, 114, 121, 126, and 131.

15. The method of claim 1, wherein the at least one Rag haplotype is a favorable Rag haplotype that positively correlates with improved soybean aphid resistance.

16. The method of claim 15, wherein the at least one favorable Rag haplotype is selected from the group consisting of Rag1-b, Rag1-c, Rag2-d, Rag3-b, and Rag3-d.

17. The method of claim 1, wherein the haplotype or marker profile is selected from the group consisting of:
(a) Rag1-b/Rag3-b;
(b) Rag1-b;
(c) Rag1-c/Rag3-d;
(d) Rag1-e; and
(j) Rag1-d/Rag2-c.

18. The method of claim 1, further comprising selecting the first soybean plant or germplasm, or selecting a progeny of the first soybean plant or germplasm.

19. The method of claim 18, further comprising crossing the selected first soybean plant or germplasm with a second soybean plant or germplasm.

20. The method of claim 19, wherein the second soybean plant or germplasm comprises an exotic soybean strain or an elite soybean strain.

21. The method of claim 18, wherein said first soybean plant or germplasm comprises a soybean variety selected from the group consisting of PI567666, PI567622, PI219652, PI219655, 95B97, PI587577E, PI587973B, PI567392, PI567055, PI567063, FC031416, PI507089B, and PI567183.

22. The method of claim 18, wherein said first soybean plant or germplasm comprises a soybean variety selected from the group consisting of PI567666, PI567622, PI219652, and PI219655.

23. An isolated polynucleotide capable of detecting: a) a marker locus selected from the group consisting of S14181-1-Q1, S13871-1-Q1, S14161-1-Q10, S09515-1-Q1, S14151-1-Q1, S14151-2-Q4, S07164-1-Q12, S14182-1-Q1, S00812-1-A, S02780-1-A, S01190-1-A, S14761-001-Q001, S14771-001-Q001, S07165-1-Q3, S14778-001-Q001, S01164-1-Q1 S13662-1-Q3/Q6, S13663-1-Q1, S11411-1-Q1, S13664-1-Q1/Q002, S13672-1-Q1/Q2/Q3, S13674-1-Q1/Q007, S13675-2-Q1; or b) a SNP loci located at a physical position selected from the group consisting of 5516385, 5516818, 5598980, 5602544, 5605203, 5605275, 5608106, 5630404, 6754454, and 6671535 on LG-M of the soybean genome, 28187733, 28829625, 28837383, 29097652, 29678319, and 29825175 on LG-F of the soybean genome; and 5140274, 5919650, 5960726, 6066531, 6231641, 6524877, and 6542422 on LG-J of the soybean genome.

24. The isolated polynucleotide of claim 23, wherein the polynucleotide comprises a nucleotide sequence selected from the group consisting of SEQ ID NOs: 1-131.

25. A kit for detecting or selecting at least one soybean plant with improved aphid resistance, the kit comprising:
a) primers or probes for detecting one or more marker loci associated with one or more quantitative trait loci associated with improved aphid resistance, wherein the primers or probes comprise an isolated nucleic acid of claim 24; and
b) instructions for using the primers or probes for detecting the one or more marker loci and correlating the detected marker loci with predicted improved resistance to aphid infestation.


SEQUENCE LISTING (131 sequences)

SEQ ID NO: 1 - DNA, 20 bp, Artificial Sequence
  Primer/Probe/Target Sequence
gcatctcatgattaagtagg

SEQ ID NO: 2 - DNA, 23 bp, Artificial Sequence
  Primer/Probe/Target Sequence
caagaactttgcttgtcttgctg

SEQ ID NO: 3 - DNA, 15 bp, Artificial Sequence
  Primer/Probe/Target Sequence
caatcagcacccttg

SEQ ID NO: 4 - DNA, 17 bp, Artificial Sequence
  Primer/Probe/Target Sequence
aagcaatcaacaccctt

SEQ ID NO: 5 - DNA, 728 bp, Glycine max
  misc_feature (28)..(28): n is a, c, g, or t
aggsaraakycacactataatggaagancacctgaatggcctatgaggcttctctaaaacagcagagaagaacatttcctgctcatccttatcaagctttggaacaaccacccatttgtgcttcttatacctcagcattttgaaggcctgcaagtaatcatacaaatctaccttcaagcagaaaaagctgtcaatccaaagcaayccccctggcctcagaactctatcccaatcatacaatataaactcaaggagcacaagatcaatccacccatcaagaaaccttgttgtgtgaatcaaatctagggtgttgtcaaaaaatggaagcctttggtttatagtcaagtaragaggaacaagtcctcttagagcaatcatttcattgaagggtgctccaaaattgatattggctgaaactatagtcacattgaattccctcatcctagcagcaaaartcccagttccaacacttaagtccaatcctataaggaaaatctgctgtgcggtctgaatcagcttccaggcttacccatcttggcatctcatgattggtgaggttgaagcaatcarcacccttgaagaatcccttacgagtggcattgccagcaagacaagcaaagttcttgcactggtattgactccatctgacatttctatcatcagggagtttccacaaggattcattgacgggaaatggttgtttgtatagcttaggggatctgaaaagc

SEQ ID NO: 6 - DNA, 20 bp, Artificial Sequence
  Primer/Probe/Target Sequence
gcaggctcatcagattgctt

SEQ ID NO: 7 - DNA, 20 bp, Artificial Sequence
  Primer/Probe/Target Sequence
gcagcgtctcatcaacaaaa

SEQ ID NO: 8 - DNA, 16 bp, Artificial Sequence
  Primer/Probe/Target Sequence
ttgaaaccaccatttt

SEQ ID NO: 9 - DNA, 15 bp, Artificial Sequence
  Primer/Probe/Target Sequence
aaactaccattttgc

SEQ ID NO: 10 - DNA, 726 bp, Glycine max
  misc_feature (658)..(658): n is a, c, g, or t
tttctatcatcagggagtttccacaaggattcattgacgggaaatggttgtttgtatagcttaggggatcttgaaaagcaccttctcctaggtaaaggatcacagccatgaaccataagcttttgagccagtttccagtcatcgttgcagatctcaccaacatcataatccatgtattcttctagctcttccttcatggcaaagcacgtgtgtcctatgctggtgaagcttgcattttcccccataaaattttgttttcctaatctgttaggtttgatcctaacatacttgcgaatctcttctcgcagaaagtagtcagcaggctcatcagattgcttcttttttgttgaaacctaccattttgctgattattgccttcattttgttgatgagacgctgccgccgactcaagaagtcctaatatatcagaaagaaatgcaccttgctggaatacagatggggtcaatgatggatcctgttttgtttccctcagtttgtccagttcctcttcaattttctgaattaccatttcaacagttttcatcagcacctcattttgattcccatttgggacctcaggattcagagattgacacaccattttagtgattgctcaacaaataaacagagataaattgacacatatcccacgcatacnaataaacagagattgacacaccattttagtgattgctcaacaaatacttaatatcaatatgcaaaaaa

SEQ ID NO: 11 - DNA, 23 bp, Artificial Sequence
  Primer/Probe/Target Sequence
caccagctcgataagctagagat

SEQ ID NO: 12 - DNA, 24 bp, Artificial Sequence
  Primer/Probe/Target Sequence
ttagccatggattttgttgaatac

SEQ ID NO: 13 - DNA, 15 bp, Artificial Sequence
  Primer/Probe/Target Sequence
ccagtagcagcccta

SEQ ID NO: 14 - DNA, 17 bp, Artificial Sequence
  Primer/Probe/Target Sequence
agtagcacccctaccaa

SEQ ID NO: 15 - DNA, 702 bp, Glycine max
  misc_feature (451)..(451): n is a, c, g, or t
agcttcgtgaagaagtataggagaggcttctctccatgaagggcggttacttccaaattgggatcattattgatgtaatttttgttagtttggatgcttgggtagtcattgggagcttagattratttgtgaatttcgcaaagtgtagaatmgcaccagctcgataagctagagattgagctagcccaccgagagcaaaccttcagagttataactagctagctaattaagccagtagcasccctaccaagcgacacattcatagsttctctttaggtttgtattcaacaaaatccatggctaagytagaggctattataaataaactvaggcgagtaaccaraatttattttagaggactagctatagcaaggcttgtggctaagccagttaccttcctgagagtgaacttgagaaagtacagaggctagctaaggtaggtattagtctncctaaacctrattatgctgaaagtctatgtttgnttaattgtntttcctccttgtttcctttggatwttttttatttgcttggtactttgtgatgttaattattgttgatawtggtannatcatatgtattttntatcatgtgattgttgcatatgcttggnattgtatgttacgatgaaccctgtaaaataaaagatacttaaaawtatacctatatctctnataaaaaggtggagagtttccttaaagg

SEQ ID NO: 16 - DNA, 25 bp, Artificial Sequence
  Primer/Probe/Target Sequence
tgcaagattgatttttatgatacgg

SEQ ID NO: 17 - DNA, 27 bp, Artificial Sequence
  Primer/Probe/Target Sequence
ggactaaaattagaaaaagaggaacca

SEQ ID NO: 18 - DNA, 17 bp, Artificial Sequence
  Primer/Probe/Target Sequence
tattgccaattcgatcc

SEQ ID NO: 19 - DNA, 16 bp, Artificial Sequence
  Primer/Probe/Target Sequence
tattgccagttcgatc

SEQ ID NO: 20 - DNA, 402 bp, Glycine max
ttgttaacacgtgataatattcattagcaactacacttagaaactaaataaatataattgacrtactattgatcatcataaaattaatattttctaattaakaaaattttgctaaagaattagctcttgctcactttgtctgatttaaccrgttctttgcaagattgatttttatgatacggaattgactggtattgccaagttcgatccggccaatttggttaagttctacttctaacaataggctaaaaatatttttttcccttgtaagttaggattttttttatttttggttcctctttttctaattttagtccttggaaattgttttttttttaattttaatccttataagatattttattatttttaattcttataaaattatatatattttttatt

SEQ ID NO: 21 - DNA, 20 bp, Artificial Sequence
  Primer/Probe/Target Sequence
ccagcttcttttgctccatc

SEQ ID NO: 22 - DNA, 21 bp, Artificial Sequence
  Primer/Probe/Target Sequence
cgacgctcctaagtattggtg

SEQ ID NO: 23 - DNA, 15 bp, Artificial Sequence
  Primer/Probe/Target Sequence
cattgtacgtccctc

SEQ ID NO: 24 - DNA, 15 bp, Artificial Sequence
  Primer/Probe/Target Sequence
atcattgtacggccc

SEQ ID NO: 25 - DNA, 714 bp, Glycine max
tttctgatatttatcatactaaacgaagcattaacctccaaatctcctcttagtaatgtattcttagaaaatagagaatctaattcatcacactctttctcatcacatactggctcggatgcttgtggcccatttcattggaaaggttctctagaaatcaagataatgtttagcaaatcaaagaacgagattctgtttgctggagcagagggagatttgtggattttctagttagcttcttcacagagaacctcttggatctattctaaaccttatgaatggcaagttatcgttgggaagcattgataacttgtgcacaagtgtgaagaatctcaacccatcatggttcatcgggtcatgaaacaaatcctttctgattaatccaagggtcgctcctaaatttggttgtaagatcaatccactaaatgttttacaagaagacgacgctcctaagtattggtgtggcactgtagcagataaagataatgagggacgtacaatgatttcaaagaaaaatgatatgctacaatatccagcaaaaaaactgaaactttttgagccaaggtgttctgatggagcaaaagaagctggtgtgggatttatgaagaggccgtgtctatttgttgtgatggatgatctgaaagtgataccaatgacaactacttctagcattgagtatctgcaaaagctggaggaggagaatgtcgagttgga

SEQ ID NO: 26 - DNA, 21 bp, Artificial Sequence
  Primer/Probe/Target Sequence
aatcccacaccagcttctttt

SEQ ID NO: 27 - DNA, 26 bp, Artificial Sequence
  Primer/Probe/Target Sequence
gtgtggcactgtagcagataaagata

SEQ ID NO: 28 - DNA, 14 bp, Artificial Sequence
  Primer/Probe/Target Sequence
cagaacatcttggc

SEQ ID NO: 29 - DNA, 14 bp, Artificial Sequence
  Primer/Probe/Target Sequence
cagaacaccttggc

SEQ ID NO: 30 - DNA, 714 bp, Glycine max
tttctgatatttakcatactaaasgaagcattaacctccaaatmtcctcttagtaatgtattcttagaaaatagagaatctaattcatcayactctttctcatcacatactggctcrgatgcttgtggcccatttcattggaaaggttctcyagaaatcaagataatgttwagcaaatcaaagarcragattctgtttgctggagcagagggagatttgtggattttctagttagcttcttcacagagaacctcttggatctattctaaaccttatgaatggcaagttatcgttgggaagcattgataacttgtgcacaagtgtgaagaatctcaacccatcatggttcatcgggtcatgaaacaaatccwtwctgattaatccaagggtcgctccyaaatttggttgtaagatcaatccactaaatgttttacaagaagacgacgctcctaagtattggtgtggcactgtagcagataaagataatgagggmcgtacaatgatttcaaagawaaatkatatgctacaatatccagcaaaaaamctgaaactttttgagccaagrtgttctgatggagcaaaagaagctggtgtgggatttatgaaraggccgtgtctatttgttgtgatggatgatctgaaagtgataccaatgacaactacttctagcattgagtatctgcaaaagctggaggaggagaatgtcgagttgga

SEQ ID NO: 31 - DNA, 22 bp, Artificial Sequence
  Primer/Probe/Target Sequence
tcatttcctgatgctcaccata

SEQ ID NO: 32 - DNA, 24 bp, Artificial Sequence
  Primer/Probe/Target Sequence
ggttgtatccatcttctgaactgc

SEQ ID NO: 33 - DNA, 17 bp, Artificial Sequence
  Primer/Probe/Target Sequence
ttgagaaaacgtctgca

SEQ ID NO: 34 - DNA, 17 bp, Artificial Sequence
  Primer/Probe/Target Sequence
ttgagaaaacatctgca

SEQ ID NO: 35 - DNA, 466 bp, Glycine max
  misc_feature (13)..(13): n is a, c, g, or t
ttatgtaacttcnttttgtagcaggctaaattatcacattgggacctacgagcgaaaaaagggtgaatctctagctatactatattattattttatttgtttcccttcactagttgatttggttgtatccatcttctgaactgcttcagatgctgcagatgctgccttgtctagacctaacctttgcaactcagttgagaaaacagtctgcatctgttttgctgattttgtatggactatggtgagcatcaggaaatgaaccatttatcacgtcttccttatactccagtaaggctttattgatgacatctcctacacgtgcatactgcttacaaaattttggagtaacctgaaaaagatacatcatctcaaaattagcaatgaattccataatgtctgaaagagaaaacaaggttaggatatgctacaaatacctttgcatggtgagggcatggtcatagctgtt

SEQ ID NO: 36 - DNA, 20 bp, Artificial Sequence
  Primer/Probe/Target Sequence
tgtactttggctgcgtctcc

SEQ ID NO: 37 - DNA, 26 bp, Artificial Sequence
  Primer/Probe/Target Sequence
ggtaactcctttgtaatgttcaccac

SEQ ID NO: 38 - DNA, 13 bp, Artificial Sequence
  Primer/Probe/Target Sequence
ccatgtcaatgcc

SEQ ID NO: 39 - DNA, 14 bp, Artificial Sequence
  Primer/Probe/Target Sequence
ccatgtcaacgcca

SEQ ID NO: 40 - DNA, 607 bp, Glycine max
actttcatcaaaagaacaaagcaaccgaattttgacacccaaatctggaattgacctgactgcaaacgacaacagtaaatattttcaatctttgaagatatagaactccaatgttactaaaaataaggctgtgaatgttagtaatggaagtaaaactaccttttctcttgatataaaaagagagtatatctcaattgatgaggatgatccagaaggcactaatgctttgcaaggatgttcaaaacacaactacaaagatcaagacagggatgatattgcttttagcaagccttcyctagtgaagctagagyctgtgtctggcataaagactgaaamatcattgcaaggaaawtgtactttggctgcgtctccyagagttgatattgacattggcrttgacatggctaacatttcagctggtgctatggatgaagatgtaactttacaaaccaacattaaacaacccgtggtgaacattacaaaggagttaccattaacactttcaaattcaggtatgctactagttgagttttaaaaacagattttttttaattaaaagtatatactattcatgatttactgaacaaaactcaaatkctgggcattt

SEQ ID NO: 41 - DNA, 24 bp, Artificial Sequence
  Primer/Probe/Target Sequence
gctgctctttctctgctgtgatca

SEQ ID NO: 42 - DNA, 26 bp, Artificial Sequence
  Primer/Probe/Target Sequence
tgggtggtttccttgtttataccaac

SEQ ID NO: 43 - DNA, 17 bp, Artificial Sequence
  Primer/Probe/Target Sequence
tatacccgtgagactat

SEQ ID NO: 44 - DNA, 17 bp, Artificial Sequence
  Primer/Probe/Target Sequence
tataccggtgagactat

SEQ ID NO: 45 - DNA, 441 bp, Glycine max
tactgaagtgaatcgatactgtattcatcaaataccgtgtaaataccaccgatctcgaawtgtctgaggtgcttgttttgagaattgtaaacatgaatwatgtatacatgtgtgattcaattaagaaaccaaataatggtattgagatggagatgctcttatcctagtgttggtggtccctttttaaaacaaaatttgcatttcaaataaactaaagatcaaaacaaacagacacgggagataaggtcttctccttctgtaaagkgcatgcaacttgctgctctttctctgctgtgatcagtgtgaagataatccaaagaaatttcaacgttttaaataaaagggtaagagttgaaactcatagtctcacsggtataaaaaaatgctagttggtataaacaaggaaaccacccacgcakggtcatagactatatacaacca

SEQ ID NO: 46 - DNA, 20 bp, Artificial Sequence
  Primer/Probe/Target Sequence
ggcatttgcttcaattttcc

SEQ ID NO: 47 - DNA, 23 bp, Artificial Sequence
  Primer/Probe/Target Sequence
acttttgcccctatakgatatgc

SEQ ID NO: 48 - DNA, 15 bp, Artificial Sequence
  Primer/Probe/Target Sequence
actctggataacctg

SEQ ID NO: 49 - DNA, 15 bp, Artificial Sequence
  Primer/Probe/Target Sequence
actctgggtaacctg

SEQ ID NO: 50 - DNA, 551 bp, Glycine max
ggttcctttgtaacttaccatctatctagaaactgcaggattctcttgtaaaataaaatattaaatgatataatacttgagatatgtagttggctaaayttcatcttatatgaggcatttgcttcaattttccagactattgcctttaccttctagactctggrtaacctgaactgcatatcmtataggggcaaaagtatgttattttgtcagcatataaacatgtttgcatcctatacagtcaagtattctacacagatactatagaaagtagaaagaatagtggtrcttttcacttgtttctgttgaaaactgaatacaaagatatagagagagtagagagaaaagggagataaggtttctctgaaaatgtctcaactctttagatgatttcgtagaggtgttcacagattggatttgattggttttgaggagtaaagttattcaatcatatcccattgtttttattttatttaaacatccaatcaatttgatcctaaattaaatatgatctaattcattccaatcaaaagtgggtttggattggatca

SEQ ID NO: 51 - DNA, 26 bp, Artificial Sequence
  Primer/Probe/Target Sequence
agagagcaacaaccagtaatttcata

SEQ ID NO: 52 - DNA, 24 bp, Artificial Sequence
  Primer/Probe/Target Sequence
acttagtgcatctattgcaaccac

SEQ ID NO: 53 - DNA, 18 bp, Artificial Sequence
  Primer/Probe/Target Sequence
ccactaaagttagcctag

SEQ ID NO: 54 - DNA, 18 bp, Artificial Sequence
  Primer/Probe/Target Sequence
ccactaaggttagcctag

SEQ ID NO: 55 - DNA, 301 bp, Glycine max
tagatctccttaatagttggttaatttttcaataatgagtgtcacaatttaccatcatagagagcaacaaccagtaatttcatattgtttaggatgaaataaaatgttttactaagatcgtaatcaaaatctaaatttgttgcccactaargttagcctagcggtagctagtggttgcaatagatgcactaagtgttacgatcgatsttcattgctattattctactaaaatcaaaatcaaaatttatttattttatatagactaaaaatatatttaaacaaattagtcatgtctgcatag

SEQ ID NO: 56 - DNA, 23 bp, Artificial Sequence
  Primer/Probe/Target Sequence
ccttcaacaacagcagctttaat

SEQ ID NO: 57 - DNA, 24 bp, Artificial Sequence
  Primer/Probe/Target Sequence
ctgcttaatcgactgagctagacc

SEQ ID NO: 58 - DNA, 18 bp, Artificial Sequence
  Primer/Probe/Target Sequence
cattagatcaaacactgc

SEQ ID NO: 59 - DNA, 18 bp, Artificial Sequence
  Primer/Probe/Target Sequence
cattagatcaaacattgc

SEQ ID NO: 60 - DNA, 301 bp, Glycine max
atgcttcattcctcccaggtatgatgttcaactaatttatttgttctagtttctgtttcatatatggaacatggaaccctctgactatcgttaaagtcagttagccttcaacaacagcagctttaatctgtgtgcacattagatcaaacaytgcatttatttcaattaatctaaacaaagaaaaaaatatgtatgtggtggtacaataaactattgtgtaacaatcaagggggtctagctcagtcgattaagcagagtatgtaagtattataaattttctggtagcgtgtttaattcctac

SEQ ID NO: 61 - DNA, 22 bp, Artificial Sequence
  Primer/Probe/Target Sequence
gcttgtaagctattcccaaacg

SEQ ID NO: 62 - DNA, 20 bp, Artificial Sequence
  Primer/Probe/Target Sequence
tatctgtgagcggttgcttg

SEQ ID NO: 63 - DNA, 19 bp, Artificial Sequence
  Primer/Probe/Target Sequence
tttcttatctaaggttttg

SEQ ID NO: 64 - DNA, 18 bp, Artificial Sequence
  Primer/Probe/Target Sequence
ttcttatcaaaggttttg

SEQ ID NO: 65 - DNA, 650 bp, Glycine max
acctgagccacactatgagctaacatgagaattcatctaatcatagtttgygtgaaatctgcttctggctacactaaataacttgaactgctctctattaatggagttaacatgaaaagagtaaattgttagcatttctttattcaagtattcatcaggtctcttttctcaagttttcattaatatggtatgaatgractgtttgttactatatctgtgagcggttgcttgcaacataaacatgtacttgctgttatgctactgctcctaaagatatgtaatttaatttagttatcaaaaccttwgataagaaaatctgcgtttgggaatagcttacaagcttcttttgtagtcgttctgacctattgtsagggtagagttacttactggtgtatatcatggttgatggatgtgtaaatttctatgcaggtcattgatgattttgctaagtttaatctgcttctcaaagtcaagaggggacagaaggaagagaagtttaaggtagaggtacacaagaataaccaaggggggttccatctaaatcagatggaacaagatcattcctaattctgatctgaagtttggtcaccctagcaatattgttcttatctatgtctagtgcttacccctttctctgtgccttggcaaat

SEQ ID NO: 66 - DNA, 26 bp, Artificial Sequence
  Primer/Probe/Target Sequence
tgaggatatttatggaatttgtcaga

SEQ ID NO: 67 - DNA, 25 bp, Artificial Sequence
  Primer/Probe/Target Sequence
catgatgagatcagaaaagaaatgc

SEQ ID NO: 68 - DNA, 17 bp, Artificial Sequence
  Primer/Probe/Target Sequence
cttataaaaccgctttc

SEQ ID NO: 69 - DNA, 18 bp, Artificial Sequence
  Primer/Probe/Target Sequence
cttataaaactgctttcc

SEQ ID NO: 70 - DNA, 301 bp, Glycine max
ccatgactgcaaattattgattgcctatttattcaatctgatcctagtggtgtagcttgttggttaagagttgaggatatttatggaatttgtcagaaaacttttcacatgtaaagttgtagtataaggaatcagacattcttataaaacygctttccatttctagctttgctggcatttcttttctgatctcatcatgatgtgaaaattatactgacatgaaattttggccactgcttgtgtgttcaaaaagctaaacttcacatacaacattttggtaacaaggttatttgtgattgca

SEQ ID NO: 71 - DNA, 21 bp, Artificial Sequence
  Primer/Probe/Target Sequence
gacagtggagagttacgagga

SEQ ID NO: 72 - DNA, 20 bp, Artificial Sequence
  Primer/Probe/Target Sequence
cacatctgaatcaccctgga

SEQ ID NO: 73 - DNA, 16 bp, Artificial Sequence
  Primer/Probe/Target Sequence
ccacctacatcactac

SEQ ID NO: 74 - DNA, 16 bp, Artificial Sequence
  Primer/Probe/Target Sequence
ccacctacatgactac

SEQ ID NO: 75 - DNA, 509 bp, Glycine max
  misc_feature (49)..(52): n is a, c, g, or t
tgtaactatatacaaaatatcacgaataactccagcaggacctaatccnnnntgattgttacatacaaacaytacaatcacttaasgaacaacaaaactmtaccagacatgatccaaaacatccttaggcacccaaaaggaatgtaagctcyaactctaacyttraaaggtcagaaggagttataagactcaccagagtcactrgacagtggagagttacgaggagaacccccaataccacctacatsactactatcaaaacctatggcttcaagccaaaaactaatccagggtgattcagatgtgtcacctttcatgaagatattgacctgcatgttaagagctcrccgcctcctgggtgtgatatgctcttggttaaggacattatggggctccytatgtcccgtwcgatttstgttcagttttcctgggcattaagccctcctcagaataaaaaaawgaaaaagaaaagaggatgtgccgcctctcgtccatggtcatagcctgtt

SEQ ID NO: 76 - DNA, 25 bp, Artificial Sequence
  Primer/Probe/Target Sequence
tctttatgatgatgagcagaagcta

SEQ ID NO: 77 - DNA, 21 bp, Artificial Sequence
  Primer/Probe/Target Sequence
caccccaaaaacaaaacactc

SEQ ID NO: 78 - DNA, 16 bp, Artificial Sequence
  Primer/Probe/Target Sequence
ctttcagagcattagc

SEQ ID NO: 79 - DNA, 16 bp, Artificial Sequence
  Primer/Probe/Target Sequence
tttgctttcagggcat

SEQ ID NO: 80 - DNA, 22 bp, Artificial Sequence
  Primer/Probe/Target Sequence
gggaagagtctgaatggtgtct

SEQ ID NO: 81 - DNA, 22 bp, Artificial Sequence
  Primer/Probe/Target Sequence
ccccaaaaacaaaacactcatc

SEQ ID NO: 82 - DNA, 16 bp, Artificial Sequence
  Primer/Probe/Target Sequence
ctttcagagcattagc

SEQ ID NO: 83 - DNA, 16 bp, Artificial Sequence
  Primer/Probe/Target Sequence
tttgctttcagggcat

SEQ ID NO: 84 - DNA, 625 bp, Glycine max
taaatatgaayatttttctcaataaaaatwaataarcttgaatcaatgttccaacacgagttacattacaacttayaaacaccatctaacgttctaagtagaggtttttgagagttatcacctggcaagaagtagctgtgtctggaaaagctgctgttggagcagaaatcacaatacacaaaacccataacactgatttgtgagatggggaacctggttttcccaggaagcttggttcttgcaggctcaacttccaaaaactgagtaaaaaagggaaaaagaagcaagagggttatggtattcatggccaactttgttaaaaaaaaatggagggaagagtctgaatggtgtctttatgatgatgagcagaagctagagcttttgctttgttttgctttgctttcagrgcattagcatttagctgctagtagaagaaaatgatgagtgttttgtttttggggtgtggataaagaggggcagtggttagtggagaaccaacttcaagcaaaaagttggaaatatttttttcttttctttttttcacaagaaacaaagacatgtgaatgttgtttggcatracatggagctcatgagtaggagagagaaggggtaagcagattytttt

SEQ ID NO: 85 - DNA, 25 bp, Artificial Sequence
  Primer/Probe/Target Sequence
tctgatgatgattatagtgggctct

SEQ ID NO: 86 - DNA, 21 bp, Artificial Sequence
  Primer/Probe/Target Sequence
tgctatgcatttgaaaccaca

SEQ ID NO: 87 - DNA, 16 bp, Artificial Sequence
  Primer/Probe/Target Sequence
ctgataacaatagccc

SEQ ID NO: 88 - DNA, 18 bp, Artificial Sequence
  Primer/Probe/Target Sequence
ataacaacagccctgact

SEQ ID NO: 89 - DNA, 752 bp, Glycine max
  misc_feature (34)..(34): n is a, c, g, or t
catgrmyttgagataggttgcaaaggcttagagnagagttatggctaaaagtgagatgaaattcatnggagtagtgattctgatgatgattatagtgggctctacacaagctgataacaayagccctgacttgactgggtgtgaagttaaatgtggtttcaaatgcatagcatatttctatagtaaaaaaaaatttgacgaatgttgtctcccttgtattcgaaaatgtcatcatgaaatgtccattgatgttgtytatgattgcattactggttgccgcttaaccaagtccattgatgacaacattggtatttatccttttgcaaaacttttattaagttttatttttataattaagttaattatgttttcacatgtttttttattattgactttacatgtcagatgctcgtgttcttaccgttcatgcaatggattcttgtgtgcaagagtgcaagaacaagtaagagtttgttgcaaaaatgctcagaaaaaaatattagttggagaaataatatgtatccatgtttgtaataaataattcttaataaatatgtaatatctcatctttattttccgtaaatcttttgtccaattatttttcttgatttgtctgatgttcctgtcatacctgagcaaataatcatgcttctaaattaaattaataatgacactaataattttattatatccaaatggaaaatgtatgcacaaaaaatattgatgaccaatatattaaaagttgagaaatt

SEQ ID NO: 90 - DNA, 21 bp, Artificial Sequence
  Primer/Probe/Target Sequence
ggacccaacatcaatcaaatg

SEQ ID NO: 91 - DNA, 20 bp, Artificial Sequence
  Primer/Probe/Target Sequence
tgcattctggaaagacatgg

SEQ ID NO: 92 - DNA, 14 bp, Artificial Sequence
  Primer/Probe/Target Sequence
ttttctgcactccc

SEQ ID NO: 93 - DNA, 14 bp, Artificial Sequence
  Primer/Probe/Target Sequence
ttttctggactccc

SEQ ID NO: 94 - DNA, 120 bp, Glycine max
ttctcatggctttctggtttgcattctggaaagacatgggcatgaaacaataatgggagtscagaaaacaacaaccccttataattctctcacaactatcatttgattgatgttgggtcc

SEQ ID NO: 95 - DNA, 21 bp, Artificial Sequence
  Primer/Probe/Target Sequence
catgccagtatgaatgtgctg

SEQ ID NO: 96 - DNA, 21 bp, Artificial Sequence
  Primer/Probe/Target Sequence
tccgcacatttagttccctta

SEQ ID NO: 97 - DNA, 18 bp, Artificial Sequence
  Primer/Probe/Target Sequence
attgtgacactctattgc

SEQ ID NO: 98 - DNA, 18 bp, Artificial Sequence
  Primer/Probe/Target Sequence
ttgtgacactctatggca

SEQ ID NO: 99 - DNA, 24 bp, Artificial Sequence
  Primer/Probe/Target Sequence
caaagtgtcatgccagtatgaatg

SEQ ID NO: 100 - DNA, 26 bp, Artificial Sequence
  Primer/Probe/Target Sequence
gttttattttcattccgcacatttag

SEQ ID NO: 101 - DNA, 18 bp, Artificial Sequence
  Primer/Probe/Target Sequence
attgtgacactctattgc

SEQ ID NO: 102 - DNA, 18 bp, Artificial Sequence
  Primer/Probe/Target Sequence
ttgtgacactctatggca

SEQ ID NO: 103 - DNA, 576 bp, Glycine max
  misc_feature (384)..(384): n is a, c, g, or t
aaaaggctatgaccatgtttttcaggggtcctggtgtaactgtactaatcctgtcatggatcatcaccctgtacactctatggcaaatggttgagatgcatgaaatggtacctggaaaacgttttgataggtatcatgaactggggcagtatgcctttggggagaagctaggactttatatagtggtgcctcaacaacttgtggtggaaattggggtgaacattgtctatatggttactgggggaaaatccttgcaaaagttccatgacactgtrtgtgacagctgcaaaaagatcaagttgacctttttcattatgatctttgcctctgttcactttgtactgtctcacctgcccaacttcaactccatttttggtgtatctntggcagcagcagttatgtccttgaggtacaagtcataaccttttagttatataggtttaattaaacttttggtcctcgttttattttcattccgcacatttagttcccttatttttttttcctacaaatttgatctyttactgttaattttgatcaatctattgtgacactctatkgcactattanntcrsm

SEQ ID NO: 104 - DNA, 20 bp, Artificial Sequence
  Primer/Probe/Target Sequence
gatcggttcccaaactagca

SEQ ID NO: 105 - DNA, 20 bp, Artificial Sequence
  Primer/Probe/Target Sequence
aacatgcaaaatgcaccaag

SEQ ID NO: 106 - DNA, 16 bp, Artificial Sequence
  Primer/Probe/Target Sequence
cagttgattactctgc

SEQ ID NO: 107 - DNA, 16 bp, Artificial Sequence
  Primer/Probe/Target Sequence
cagttgattactttgc

SEQ ID NO: 108 - DNA, 20 bp, Artificial Sequence
  Primer/Probe/Target Sequence
cggttcccaaactagcaggt

SEQ ID NO: 109 - DNA, 22 bp, Artificial Sequence
  Primer/Probe/Target Sequence
tgcaaaatgcaccaagttagat

SEQ ID NO: 110 - DNA, 22 bp, Artificial Sequence
  Primer/Probe/Target Sequence
agatcggttcccaaactagcag

SEQ ID NO: 111 - DNA, 21 bp, Artificial Sequence
  Primer/Probe/Target Sequence
catgcaaaatgcaccaagtta

SEQ ID NO: 112 - DNA, 16 bp, Artificial Sequence
  Primer/Probe/Target Sequence
cagttgattactctgc

SEQ ID NO: 113 - DNA, 16 bp, Artificial Sequence
  Primer/Probe/Target Sequence
cagttgattactttgc

SEQ ID NO: 114 - DNA, 480 bp, Glycine max
tccatctaagtctacaactctttccacatcttagaagaccttcaccaacatgcaaaatgcaccaagttagatacatatatatcatatcataccccttaatttattgcaragtaatcaactgtagaacatgtgaacacgacttttaataaactaacttattacctgctagtttgggaaccgatctccagcaatggataccattgttgagaataaagttcatgagcttgtgatcctcctcaatagtccatggacctctcttcaaaccaaccttatcacaacaaggttgtcttcccattttgtaagccttttcttttctttgagaaatcaagacaaatatatcccttgatatagaaagttactaatawcaagtgtgaaacmttggaagcatgacacttaaaaaggagcttgagattgaagcaaagatgctgccagttcattcaaacatttaaataaaagcatgatgtcctttgaggggggaga

SEQ ID NO: 115 - DNA, 20 bp, Artificial Sequence
  Primer/Probe/Target Sequence
ccaccattacccctctcctt

SEQ ID NO: 116 - DNA, 22 bp, Artificial Sequence
  Primer/Probe/Target Sequence
acctagcattgcaatctcttcc

SEQ ID NO: 117 - DNA, 14 bp, Artificial Sequence
  Primer/Probe/Target Sequence
ttggcattcagccc

SEQ ID NO: 118 - DNA, 15 bp, Artificial Sequence
  Primer/Probe/Target Sequence
tttggcattcacccc

SEQ ID NO: 119 - DNA, 25 bp, Artificial Sequence
  Primer/Probe/Target Sequence
ttacccctctcctttctcaacatta

SEQ ID NO: 120 - DNA, 24 bp, Artificial Sequence
  Primer/Probe/Target Sequence
tgcaatctcttccaagctagaact

SEQ ID NO: 121 - DNA, 763 bp, Glycine max
  misc_feature (17)..(17): n is a, c, g, or t
atctatcttcaatccanatctacaaaaacttagatccacaaaatmmanatatgcaaaaccaaaaatgacgatggtgaatggtggtgtcgcgatccaaaacccgtggcggtggtttaacggaatggtggtgggggaaaactggtaagggttcacactttcacgttgggtaccaaaagggcacaaatctggttctatggggggggcattttagttctccgtcggtggtgaagggtggtgtcacaatggcaagcgtgtctggttttttttcactgtcgcttcgagaatcccgtcaatttgctcttcactatcgctttcagatttcactcatagtgcattctttctgtcgatgtgtcactctccacctagcattgcaatctcttccaagctagaactagaatttagatgtgcatatctcaaagtttgaagcagaaatctgagacgatgggstgaatgccaaattttttgttgcaataatgttgagaaaggagaggggtaatggtgggcaggggttgaaaaactgaaaaattgagtgtaaaccgaaaaccaaccaaaaaaccacaaactacaaaaaaaatcgaagagcattgatttagtttggtttttgttttcccaaccgagcgggttgattcaatttttggtttaacatgcaaaaattgaaycaaaccaaatcgaactgcactygttagtttaagtttaattattataggactcaagactcatgcatactacatgtagtttaagtttaagcttatgnntnaacgac

SEQ ID NO: 122 - DNA, 20 bp, Artificial Sequence
  Primer/Probe/Target Sequence
aggtggtggcagtgttgatt

SEQ ID NO: 123 - DNA, 20 bp, Artificial Sequence
  Primer/Probe/Target Sequence
ctccaacatggctgtgctaa

SEQ ID NO: 124 - DNA, 14 bp, Artificial Sequence
  Primer/Probe/Target Sequence
aaccgtggctcatt

SEQ ID NO: 125 - DNA, 15 bp, Artificial Sequence
  Primer/Probe/Target Sequence
caaaccgtggcttat

SEQ ID NO: 126 - DNA, 467 bp, Glycine max
yggaaaatgataagcccccaacctgtgtttgttgggggaaaattaggtgmatttctgtactggtaattggttttaaattttaatccaatttcccttattctgttactagattaaagggattgacagacatgcaaaaatgtggaggaaaaggtgtgtttttcagagcatttgggccttcctaacttgatttcagcccctggacctccaacatggctgtgctaaagccttggagcaccttggaaaaatatctttgtttggaaatagcatgtttgtgctagaatttacatgactgcrtttaatagagccacggtttggttattaatcaacactgccaccacctgcccctgtttttgtaattctgactcagttgtgtgctatttaagcctgtcaattgacctagggctggacttttggttgctgttgacacattctraasgttttcttgagttctcaaccgttttctaggg

SEQ ID NO: 127 - DNA, 20 bp, Artificial Sequence
  Primer/Probe/Target Sequence
ttcagctccccattatttcg

SEQ ID NO: 128 - DNA, 20 bp, Artificial Sequence
  Primer/Probe/Target Sequence
ttggccaacctatcctcaac

SEQ ID NO: 129 - DNA, 15 bp, Artificial Sequence
  Primer/Probe/Target Sequence
tcagctcatttttgt

SEQ ID NO: 130 - DNA, 14 bp, Artificial Sequence
  Primer/Probe/Target Sequence
cagctcacttttgt

SEQ ID NO: 131 - DNA, 548 bp, Glycine max
  misc_feature (13)..(13): n is a, c, g, or t
tggcttctagganataacacatgttatttgaaggaataataataatcctactcannnatnnagtcgacttcagctccccattatttcgcagctccatcacaatatcatgaccaccaatcagctcayttttgtagtagagttgaggataggttggccaatttgagtataccttcaatccctgtctcacttcctcatcagtcaatatatcaaaggacccaaaattcaagccctcttgtcgaagggcatcagcaactctggaactaaaaccacatcttggtgcatctggggtacccttcatgaacagcatcacaggggacgaggcaatcaaattcttcagtcgatcttgaatggtctctgcaggaagaatccctttctcgtgtaaattcttcytaagttctccgcttttttgcatctccaacacaatatctgatccaccaataagctcacccttgatatacagttgaggataactggaccagtttgaataaaccttaagcccttgacgaacttcttcatcagtaagaatgtcaaaactctcaaagggaaca